(19) United States
(12) Patent Application Publication
(10) Pub. No.: US 2007/0048340 A1
(43) Pub. Date: Mar. 1, 2007

(54) Multi step patterning of a skin surface

(75) Inventors: Bran Ferren, Beverly Hills, CA (US); Muriel Y. Ishikawa, Livermore, CA (US); Edward K.Y. Jung, Bellevue, WA (US); Nathan P. Myhrvold, Medina, WA (US); Lowell L. Wood JR., Livermore, CA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/217,111

(22) Filed: Aug. 31, 2005

(51) Int. Cl. A61K 8/02

(52) U.S. Cl. 424/401

(57) ABSTRACT

Methods and systems for treating skin for aesthetic or health or other purposes are described. According to various embodiments, photoresponsive materials and light are delivered in controlled fashions to produce a patterned distribution of one or more material in or upon the skin.

### CROSS-REFERENCE TO RELATED APPLICATIONS

The present application is related to, claims the earliest available effective filing date(s) from (e.g., claims earliest available priority dates for other than provisional patent applications; claims benefits under 35 USC § 119(e) for provisional patent applications), and incorporates by reference in its entirety all subject matter of the following listed application(s) (the “Related Applications”) to the extent such subject matter is not inconsistent herewith; the present application also claims the earliest available effective filing date(s) from, and also incorporates by reference in its entirety all subject matter of any and all parent, grandparent, great-grandparent, etc. applications of the Related Application(s) to the extent such subject matter is not inconsistent herewith. The United States Patent Office (USPTO) has published a notice to the effect that the USPTO's computer programs require that patent applicants reference both a serial number and indicate whether an application is a continuation or continuation in part. The present applicant entity has provided below a specific reference to the application(s) from which priority is being claimed as recited by statute. Applicant entity understands that the statute is unambiguous in its specific reference language and does not require either a serial number or any characterization such as “continuation” or “continuation-in-part.” Notwithstanding the foregoing, applicant entity understands that the USPTO's computer programs have certain data entry requirements, and hence applicant entity is designating the present application as a continuation in part of its parent applications, but expressly points out that such designations are not to be construed in any way as any type of commentary and/or admission as to whether or not the present application contains any new matter in addition to the matter of its parent application(s).

### RELATED APPLICATIONS

1. For purposes of the USPTO extra-statutory requirements, the present application constitutes a continuation in part of currently co-pending United States patent application entitled METHOD AND SYSTEM FOR TEMPORARY HAIR REMOVAL, naming Bran Ferren, Muriel Y. Ishikawa, Edward K. Y. Jung, Nathan P. Myhrvold, Clarence T. Tegreene, and Lowell L. Wood, Jr. as inventors, U.S. application Ser. No. 11/073,361, filed Mar. 4, 2005.

2. For purposes of the USPTO extra-statutory requirements, the present application constitutes a continuation in part of currently co-pending United States patent application entitled HAIR TREATMENT SYSTEM, naming Bran Ferren, Muriel Y. Ishikawa, Edward K. Y. Jung, Nathan P. Myhrvold, Clarence T. Tegreene, and Lowell L. Wood, Jr. as inventors, U.S. application Ser. No. 11/072,698, filed Mar. 4, 2005.

3. For purposes of the USPTO extra-statutory requirements, the present application constitutes a continuation in part of currently co-pending United States patent application entitled HAIR REMOVAL SYSTEM WITH LIGHT SOURCE ARRAY, naming Bran Ferren, Muriel Y. Ishikawa, Edward K. Y. Jung, Nathan P. Myhrvold, Clarence T. Tegreene, and Lowell L. Wood, Jr. as inventors, U.S. application Ser. No. 11/072,007, filed Mar. 4, 2005

4. For purposes of the USPTO extra-statutory requirements, the present application constitutes a continuation in part of currently co-pending United States patent application entitled SKIN TREATMENT INCLUDING PATTERNED LIGHT, naming Bran Ferren, Muriel Y. Ishikawa, Edward K. Y. Jung, Nathan P. Myhrvold, and Lowell L. Wood, Jr. as inventors, U.S. application Ser. No. 11/143,925, filed Jun. 2, 2005.

5. For purposes of the USPTO extra-statutory requirements, the present application constitutes a continuation in part of currently co-pending United States patent application entitled PHOTOPATTERNING OF SKIN, naming Bran Ferren, Muriel Y. Ishikawa, Edward K. Y. Jung, Nathan P. Myhrvold, and Lowell L. Wood, Jr. as inventors, U.S. application Ser. No. 11/143,116, filed Jun. 2, 2005.

6. For purposes of the USPTO extra-statutory requirements, the present application constitutes a continuation in part of currently co-pending United States patent application entitled HAIR MODIFICATION USING CONVERGING LIGHT, naming Bran Ferren, Muriel Y. Ishikawa, Edward K. Y. Jung, Nathan P. Myhrvold, Clarence T. Tegreene, and Lowell L. Wood, Jr. as inventors, U.S. application Ser. No. 11/171,649, filed Jun. 29, 2005.

7. For purposes of the USPTO extra-statutory requirements, the present application constitutes a continuation in part of currently co-pending United States patent application entitled MULTI STEP PHOTOPATTERNING OF SKIN, naming Bran Ferren, Muriel Y. Ishikawa, Edward K. Y. Jung, Nathan P. Myhrvold, Lowell L. Wood, Jr., and Victoria Y. H. Wood as inventors, U.S. application Ser. No. 11/175,984 filed Jul. 5, 2005.

8. For purposes of the USPTO extra-statutory requirements, the present application constitutes a continuation in part of currently co-pending United States patent application entitled HOLOGRAPHIC TATTOO, naming Bran Ferren, Muriel Y. Ishikawa, Edward K. Y. Jung, Nathan P. Myhrvold, Lowell L. Wood, Jr., and Victoria Y. H. Wood as inventors, U.S. application Ser. No. 11/198,910, filed Aug. 5, 2005.

### TECHNICAL FIELD

The present application relates, in general, to the field of treating skin for aesthetic and/or health and/or other purposes. In particular, this application relates to methods and systems for controlling the delivery of materials into or onto skin.

### BACKGROUND

The introduction of various dyes or other pigmented materials into or onto the skin in the form of cosmetics or tattoos is well known, as is the application of various biologically active compounds onto or into the skin surface for various medical-related purposes. In recent years, light-activated photodynamic therapy agents have been developed for the treatment of various skin problems, including skin cancers.

### SUMMARY

According to various embodiments, methods are provided for forming patterned distributions of materials on the skin of a subject. A desired pattern may be formed by delivering a photoresponsive material to the skin and exposing the skin to light or other electromagnetic energy to cause a reaction or conversion of the photoresponsive material. In some embodiments, a photoresponsive material may be delivered into or onto the skin in a pattern. In some embodiments, patterned light may be delivered to the skin. One or both the photoresponsive material and light may be patterned in order to form a desired distribution of material. Materials distributed in or on the skin may have a variety of properties for aesthetic, cosmetic, functional, health, or medical purposes. Features of various embodiments will be apparent from the following detailed description and associated drawings.

### BRIEF DESCRIPTION OF THE FIGURES

Features of the invention are set forth in the appended claims. The exemplary embodiments may best be understood by making reference to the following description taken in conjunction with the accompanying drawings. In the figures, like referenced numerals identify like elements.

FIG. 1 illustrates focusing of light in a skin region to produce modification of a photoresponsive material;

FIG. 2A illustrates transformation of a photoresponsive substance from a first form to a second form with exposure to light;

FIG. 2B illustrates cross-linking of a photoresponsive substance on exposure to light;

FIGS. 3A-3C illustrate photopatterning of skin by targeted application of light;

FIG. 4A illustrates topical application of a photoresponsive material;

FIG. 4B illustrates diffusion of topically applied photoresponsive material into the skin;

FIG. 5A illustrates hypodermal injection of photoresponsive material;

FIG. 5B illustrates diffusion of injected photoresponsive material;

FIG. 6 illustrates injection of photoresponsive material into skin with a microneedle array;

FIG. 7 depicts diffusion of photoresponsive material into skin from a capillary;

FIG. 8 depicts a skin region including a photoresponsive substance;

FIG. 9 depicts targeted application of light to a skin region including a photoresponsive substance;

FIG. 10 depicts an embodiment of a system for controlled delivery of light to skin;

FIG. 11 is a flow diagram of a method of forming a pattern in a skin volume;

FIG. 12 is a flow diagram of a further method of forming a pattern in skin;

FIG. 13 is a flow diagram of a further method of forming a pattern in skin;

FIG. 14 is a block diagram of a system for targeted application of light to skin;

FIG. 15 is a block diagram of a system for targeted application of light to skin;

FIG. 16 is a block diagram of an embodiment of a system for controlled delivery of light to skin;

FIG. 17 is a flow diagram of a method producing a pattern on a surface;

FIGS. 18A-18D depict steps of a method of patterning skin;

FIG. 19A illustrates an embodiment of a mask with a decorative pattern;

FIG. 19B depicts use of the mask depicted in FIG. 19A;

FIG. 19C illustrates a decorative pattern formed on a skin surface with the use of the mask depicted in FIG. 19A;

FIG. 20 is a flow diagram of a method of forming a patterned distribution of material in skin;

FIG. 21A illustrates delivery of patterned light to a treated skin surface;

FIG. 21B illustrates a pattern formed on a skin surface by the patterned light depicted in FIG. 21A;

FIG. 22 is a flow diagram illustrating variations of methods for photopatterning of skin;

FIGS. 23A-23C illustrate steps of forming a patterned distribution of material in skin;

FIG. 24 is a flow diagram illustrating variations of methods for photopatterning of skin;

FIGS. 25A-25B illustrate patterning of skin by patterned delivery of photoresponsive material combined with patterned delivery of light;

FIG. 26 is a block diagram of a system for photopatterning of skin;

FIG. 27 is a flow diagram of a method of photopatterning skin including reversing the photoreaction;

FIG. 28 is a flow diagram of a method of photopatterning skin including removing the modified form of the photoresponsive material;

FIG. 29 is a flow diagram of a method of photopatterning skin including removing unmodified photoresponsive material from the skin;

FIG. 30 is a flow diagram of a method of photopatterning an active chemical compound in the skin;

FIG. 31 is a flow diagram of a method of manufacturing a device for delivering patterned light;

FIG. 32 is a flow diagram of a further method of manufacturing a device for delivering patterned light;

FIG. 33 is a block diagram of a system for delivery of patterned light;

FIGS. 34A and 34B illustrate a mounting system for maintaining alignment of masks;

FIGS. 35A-35C illustrate the use of indicia marked on the skin for maintaining alignment of masks;

FIGS. 36A-36G illustrate a multi step method for photopatterning of skin;

FIG. 37 depicts steps of a multi step method for photopatterning of skin;

FIG. 38 depicts steps of a further multi step method for photopatterning of skin

FIG. 39 is a flow diagram of a method of forming a multi-layer structure on skin;

FIG. 40 is a flow diagram of a method of forming a patterned distribution of material on a skin surface region;

FIG. 41 is a flow diagram of a method of modifying a skin surface region;

FIG. 42 illustrates a dam surrounding a photoresponsive material on a skin region;

FIG. 43 illustrates a patch including a photoresponsive material on a skin region;

FIG. 44 illustrates an envelope containing a photoresponsive material on a skin region;

FIG. 45A is a cross-sectional view of a dam containing a photoresponsive material on a skin region;

FIG. 45B is a cross-sectional view the skin region of FIG. 45 A, following removal of the dam and photoresponsive material;

FIG. 46A is a cross-sectional view of a further embodiment including a dam containing a photoresponsive material on a skin surface;

FIG. 46B depicts the embodiment of FIG. 46A following removal of the dam and photoresponsive material;

FIG. 47 is a cross-sectional view of a patch including a photoresponsive material on a skin region;

FIG. 48A is a cross-sectional view of an envelope containing a photoresponsive material on a skin region;

FIG. 48B is a cross-sectional view of the skin region of FIG. 48A, following removal of portions of the envelope and photoresponsive material;

FIG. 49 is a cross-sectional view of a rough skin surface that has been smoothed by formation of a multi-layer structure on the skin surface;

FIG. 50 is a cross-sectional view of a rough skin surface including a smoothing layer and a multi-layer structure;

FIG. 51A is a cross-sectional view of a rough skin surface region;

FIG. 51B is a cross-sectional view of the skin surface region of FIG. 51A following a smoothing step; and

FIG. 51C is a cross-sectional view of the smoothed skin surface region of FIG. 51B including a multi-layer structure.

### DETAILED DESCRIPTION

In the following detailed description, reference is made to the accompanying drawings, which form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined by the appended claims.

Throughout the specification and claims, the following terms take the meanings explicitly associated herein unless the context dictates otherwise. The meaning of “a”, “an”, and “the” include plural references. The meaning of “in” includes “in,” “immediately proximate to” and “on.” A reference to the singular includes a reference to the plural unless otherwise stated or inconsistent with the disclosure herein.

According to various embodiments as disclosed herein, methods and systems are provided for forming patterned distributions of materials in or on skin. Patterned distributions of materials in skin may have various applications, including but not limited to commercial, aesthetic, cosmetic, structural, medical or health purposes. Patterned distributions of light modulating materials such as dyes, pigments, or other light-absorbing, -reflecting, -scattering, -polarizing, -dispersing, -diffracting, -fluorescing, -phosphorescing or -emitting materials, (or any other materials that may produce a visually or optically detectable effect) may be used for aesthetic, decorative, commercial, political or cosmetic purposes (for example, as tattoos or permanent or semi-permanent cosmetics, or for commercial-speech or political-advocacy purposes). Detectable markings, which may be detectable visually or optically (e.g. at various wavelengths, not necessarily within the visible spectrum), or by electrical, magnetic, acoustic, or various other detection methods, may have functional applications, as well, for example, marking the location of a surgical site on a patient, or for providing permanent or semi-permanent identifying markings, e.g., on pets, livestock, etc. The term optical, as used herein, can refer or pertain to the use or manipulation of light or electromagnetic radiation not only within the visible portions of the spectrum, but also within the near- and far-ultraviolet and near- and far-IR portions of the spectrum. Patterned distributions of materials having pharmaceutical activity or medical significance may be used to selectively treat or aid the treatment of various structures in or near the skin surface. Treatment targets may include skin lesions, including cancerous and precancerous skin lesions, moles, warts, and sites-of-infection such as ‘pimples’. Treatment may also be applied to disorders of various skin structures, for example, capillaries, veins, arteries, other vascular components, peripheral nervous system components, sweat glands, and hair follicles and components thereof. Patterned distributions of materials that modulate physiological processes of various types (e.g., melanin production, hair growth, oil production) may be formed; for example. In other embodiments, patterned distributions of structural materials (e.g., materials that add strength, form, shape, bulk, resilience, or other desired structural or mechanical properties to skin, connective tissue, cartilage, and so forth) may be used for cosmetic or reconstructive surgery applications. In some cases, a few examples of which are provided above, it may be desirable to form a pattern of material that remains in the skin for a predictable interval-of-time, permanently or semi-permanently. In other cases, e.g., if the patterned material is a biologically active compound intended to treat a specific medical problem, only transient presence of the patterned material may be desired or may be sufficient for the desired purpose. According to various embodiments described herein, patterned distributions of material may be formed within the skin or on the skin surface.

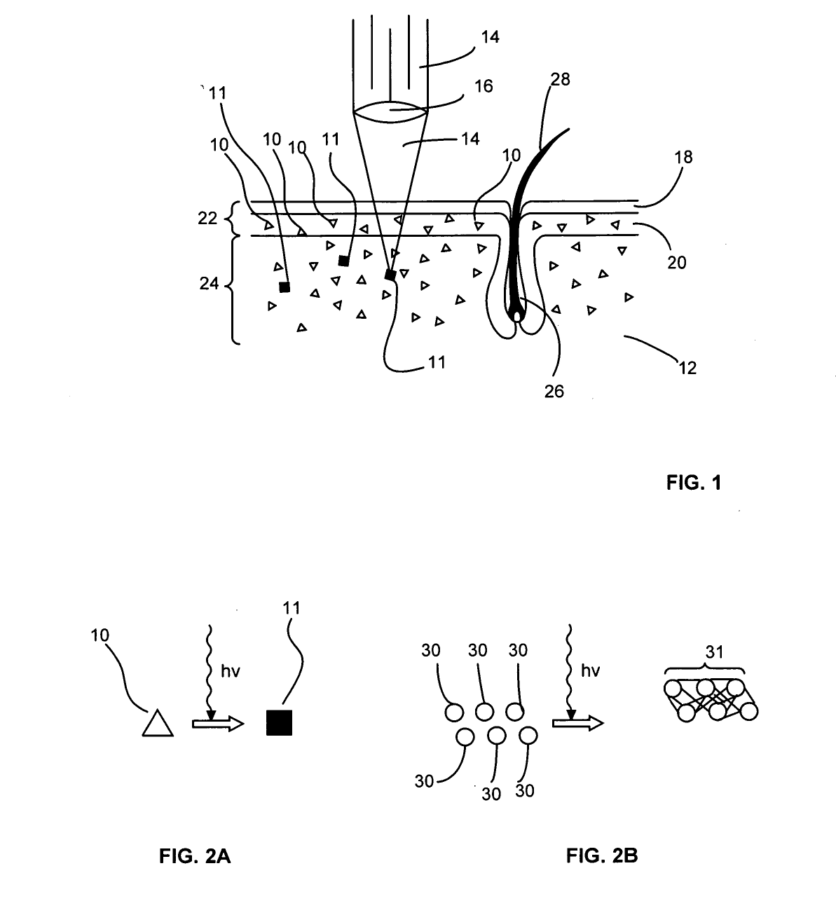

FIG. 1 illustrates modification of a photoresponsive material in skin caused by delivery of light. In FIG. 1, molecules or particles of photoresponsive material 10 are distributed throughout skin region 12, and light 14 is targeted to a specific location by lens 16, where it produces a reaction or other modification of one or more molecules or particles of photoresponsive material 10 to produce modified form 11. Skin region 12 includes stratum corneum 18 and keratinocyte layer 20, which together form epidermis 22, and dermis 24. Also shown is hair follicle 26 and hair 28. Photoresponsive material 10 may be distributed in the form of molecules, clusters or aggregations of molecules, particles, gels, solutions, emulsions, suspensions, sprays, fluids, powders, among others. As used herein, the term photoresponsive material refers to a material (compound, element, composite material, mixture of compounds or substances, etc.) that undergoes or participates in a reaction, interaction, transformation, modification, phase change, change in energetic state, etc. in response to exposure to light to produce at least one reaction product, or modified form, indicated by reference number 11 in FIG. 1, having one or more different activities or properties than the original or ‘unmodified’ photoresponsive material. A “modification”, as used herein, may include chemical reactions, changes in energetic state, phase, conformation, associations, aggregations, formation of bonds or other interactions (e.g. molecular bonds, hydrogen bonds, van der Waals linkages, etc.), polymerization, cross-linking, dimerization, breaking of bonds (e.g. by a photolytic reaction), dissociation of associated molecules, atoms, ions, etc., oxidation or reduction reactions, formation of ions or free radicals, changes of 3-D molecular structure, for example. Photoresponsive material may be any material that is responsive, reactive, or sensitive to light to change from a first state to a second state, by itself or in cooperation or reaction with other materials naturally or deliberately made to be present. Photoresponsive materials may undergo photochromic reactions, changes in luminescent behavior, magnetic interactions of metal sites, metal-ligand coordinations by photoisomerization, for example. Exposure to light may modify structural or light-modulating properties of photoresponsive materials, or both. As used herein, photoresponsive materials may react to light in the presence of a catalyst, or catalyze the reaction of other materials in the presence of light. Photoresponsive materials may respond directly to external light delivered to the skin, or respond indirectly to externally delivered light by responding to an effect produced within the skin by the light. In some embodiments, a photoresponsive material may undergo a modification that results in a modification to a secondary material, in which it is the secondary material that produces an effect in the skin. In other embodiments, the photoresponsive material may be employed as a light-specified ‘mask’ which then is used to control the exposure of skin not so ‘masked’ to subsequent processing. Photoresponsive material may include mixtures of materials that react or interact upon exposure to light. Different components of a photoresponsive material may respond to light of different wavelengths, polarities, intensity, and so forth. FIG. 2A depicts a change in conformation produced by exposure to light, in which photoresponsive material 10 is converted from a first state 10 to a second state 11. FIG. 2B depicts cross-linking of multiple molecules 30 of photoresponsive material produced by exposure to light, to form cross-linked network 31. Conversion of a photoresponsive material from an unreacted to a reacted form may include conversion from inactive to active form, from active to inactive form, from colored form to non-colored form (or vice versa), from a darker (less reflective or emissive) form to a lighter (more reflective or emissive) form (or vice versa), from a more-scattering form to a less-scattering form (or vice versa), from a first color to a second color, or any combination of these. Conversion of a photoresponsive material from an unreacted form to a reacted form may include a change in the scattering or absorption properties of the photoresponsive material for light of a given waveband.

Various methods of delivering photoresponsive material and light to a skin region may be used to produce a patterned distribution of a material in the skin region. One or the other or both of the photoresponsive material and the light may be delivered in a targeted or spatially-varying fashion in order to produce a patterned distribution of material in the skin, including a patterned distribution having no obviously-ordered features, e.g. one that appears to be ‘random’.

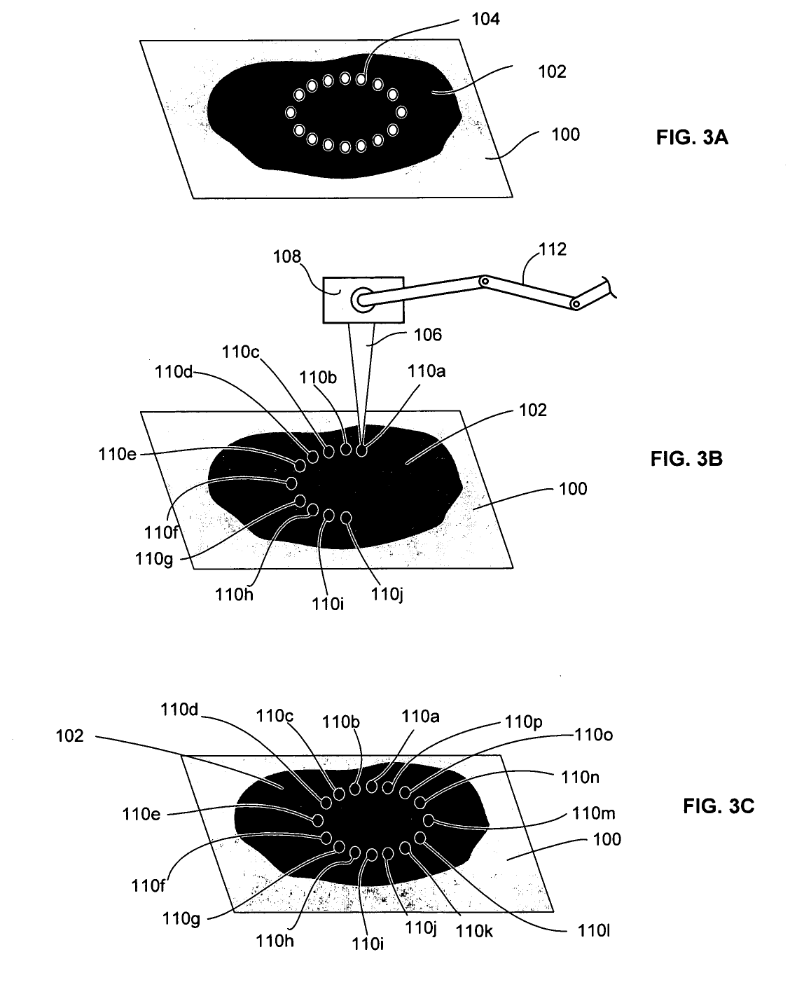

In some embodiments, a patterned distribution of a material in or on skin may be produced by delivering a photoresponsive material to at least a skin region of a subject in a relatively non-targeted fashion, and delivering targeted light to the skin region according to a pattern. The targeted light may have a wavelength content, time-averaged flux and/or fluence sufficient to cause a transformation of the photoresponsive material to a modified form, as a function of spatial position in or on the skin. As illustrated in FIGS. 3A-3C, the method may include delivering targeted light to the skin region according to a pattern by delivering targeted light to a plurality of locations in the skin region according to a pattern. A patterned distribution of the modified form of the photoresponsive material may then be formed. This general approach is illustrated in FIG. 3A-3C. In FIG. 3A, a skin region 100 is illustrated. Photoresponsive material has been applied to a portion 102 of skin region 100. Locations at which light is to be delivered to produce modification of the photoresponsive material are represented by white circles in this figure, as indicated by reference number 104. Focused light 106 from light source 108 is delivered to location 110a, which is one of multiple locations 110a-110j within portion 102 in FIG. 3B. FIG. 3B illustrates delivery of light 106 to location 110a, where photoresponsive material is converted to a modified form, indicated by a dark circle. FIG. 3B depicts multiple locations 110b-110j that have previously been exposed to light to cause modification of photoresponsive material. Light source 108 may be positioned with respect to skin region 100 by a linkage 112. FIG. 3C depicts a pattern of modified material at locations 110a-110p.

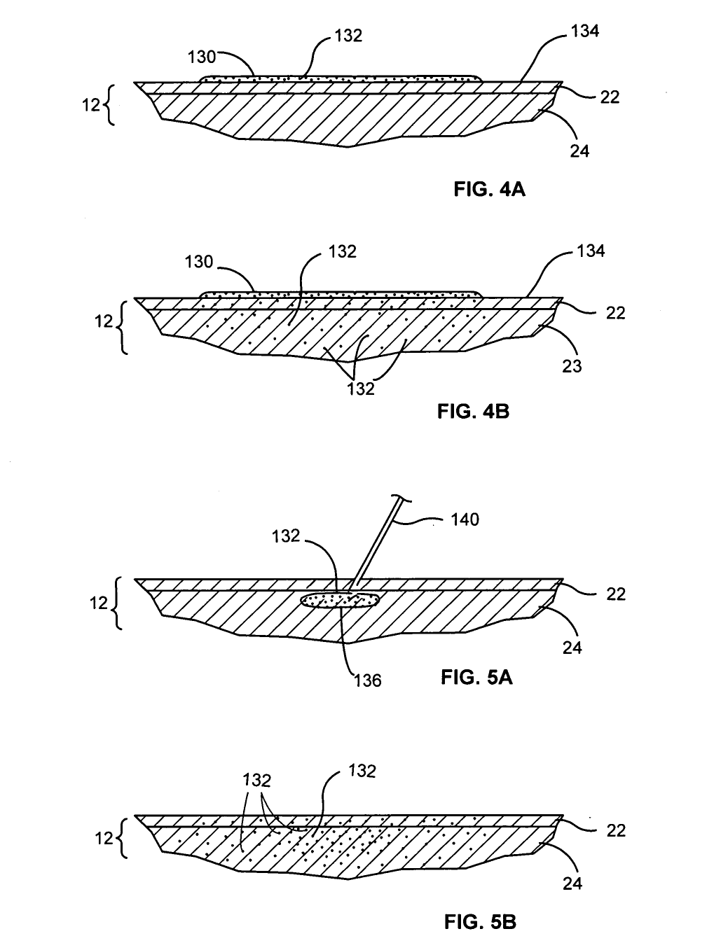

Delivery of photoresponsive material in a relatively non-targeted fashion may be accomplished by various methods, which may depend on various factors, including the type of photoresponsive material to be used, the desired depth of delivery of the material in the skin, or the size of the area in which a patterned distribution of material is to be produced. In some embodiments, photoresponsive material may be delivered to the skin topically. As illustrated in FIG. 4A, a carrier material 130 containing a photoresponsive material 132 may be placed on a skin surface 134. Photoresponsive material 132 may diffuse out of carrier material 130 and into skin 12, as shown in FIG. 4B. Skin 12 includes epidermis 22 and dermis 24. Diffusion of photoresponsive material 132 may be enhanced by electrophoresis or by the presence of solvent or ‘carrier’ chemicals such as DMSO or EDTA in certain embodiments (see, e.g., “Photodynamic Therapy”, Medscape Dermatology 3(2), 2002, incorporated herein by reference. Other methods for enhancing movement of materials into the skin may include ultrasonic-transducer-driven pressure waves, for example. Photoresponsive material may be delivered to at least a skin region of a subject topically in various forms, including, for example, an aerosol, cream, emulsion, gel, liquid, vapor, gas, lotion, patch, or powder or combinations of these.

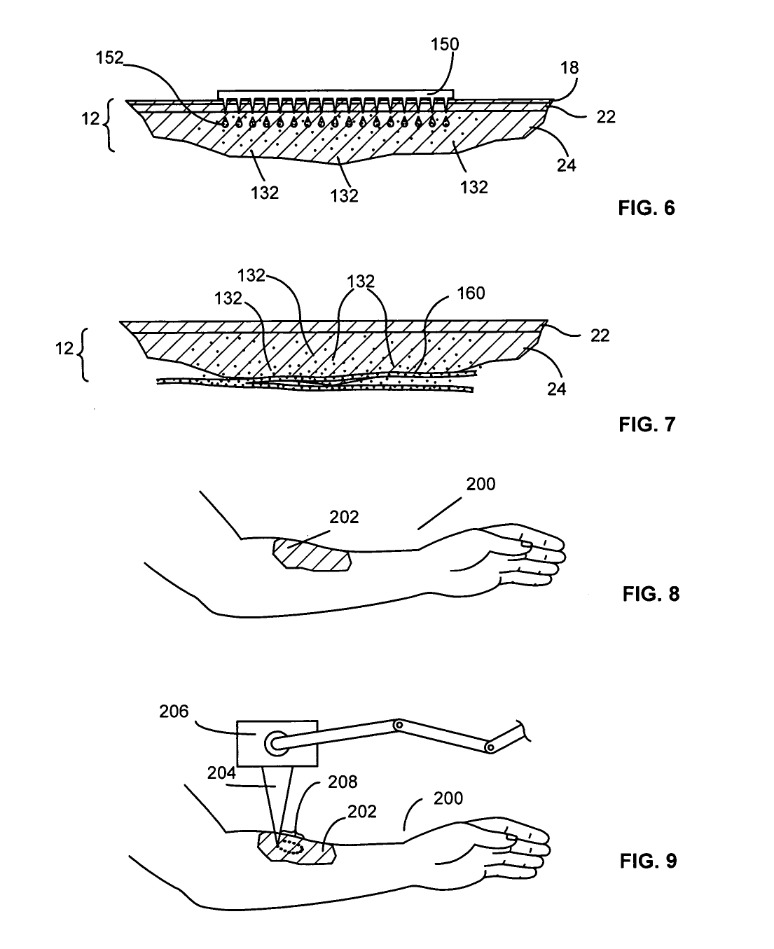

In some cases, a general distribution of a photoresponsive material within a skin region may be obtained by injecting the photoresponsive material 132 into skin 12 with an hypodermic needle 140, as depicted in FIG. 5A. Photoresponsive material 132 may be in a liquid carrier solution 136, or in a suspension, an emulsion, or any other form suitable for delivery via a hypodermic needle. This approach may be suitable if the diffusion or dispersion of the photoresponsive material away from the injection site produces an acceptable (e.g., sufficiently uniform) distribution of photoresponsive material, as depicted in FIG. 5B, within an acceptable amount of time. Alternatively, photoresponsive material may be distributed into a skin region 12 with the use of a microneedle array 150, as depicted in FIG. 6. Photoresponsive material 132 may be injected below stratum corneum 18 of skin region 12 with the use of a microneedle array 150. As described in connection with the embodiment depicted in FIG. 5A, photoresponsive material to be delivered via microneedle array 150 may be carried in a carrier fluid 152 that is adapted for use with a microneedle array. Alternatively, one or more high pressure jets or microjetted stream of fluid may be employed for delivering materials into the skin.

The distribution of photoresponsive material 132 that can be obtained within skin region 12 may depend on the combination of injection methodology and photoresponsive material used. For example, smaller molecules may diffuse or disperse more readily from the injection site than may larger molecules. In addition, the presence of certain functional groups may cause some photoresponsive materials to be taken up or retained or processed by certain tissues or cell types. Accordingly, photoresponsive materials may be selected or designed for use in combination with certain delivery mechanism and for preferential delivery to, retention by, or processing by certain tissues or cells. The design or selection of photoresponsive materials to have certain diffusion or selective uptake-or-retention-or-processing properties may be performed by a person of skill in the relevant art, for example, as described in Pogue and Hasan, “Targeting in Photodynamic Therapy and Photo-Imaging, Optics & Photonics News, August 2003, pp. 36-43, which is incorporated herein by reference.

In some embodiments, a photoresponsive material may be delivered to at least a skin region of a subject by delivering the photoresponsive material to the subject systemically. For example, photoresponsive material may be delivered to the subject orally in an ingestible formulation, via an inhalant, via intravenous or other ‘deep’ injection modalities or via various other regional or systemic routes. In some cases, a photoresponsive material may be delivered via injection, but subsequently carried throughout the body by the blood stream. As depicted in FIG. 7, a systemically delivered photoresponsive material 132 may be carried in the blood stream (e.g., in capillary 160) and diffuse out into the skin region of interest, which in this example is skin region 12. Depending on the particular photoresponsive material, it may distribute uniformly throughout the subject's body, or may distribute preferentially to certain regions, tissues, or cells of the body. In this, and other embodiments, the photoresponsive material may be attached to a carrier molecule compounded in various ways as known to those of skill in the arts of drug delivery, in order to produce a desired distribution of photoresponsive material within the subject's body.

FIG. 8 depicts the arm 200 of a subject, showing a skin region 202 in which a photoresponsive material is distributed. In this and other embodiments, photoresponsive material may be distributed only to the skin region of interest (skin region 202 in the present example), by, for example, topical application or local injection, or it may be distributed to a larger portion of the subject's body (up to and including the entire body), of which the region of interest is a part. In FIG. 9, patterned light 204 is delivered to skin region 202 from light source 206 to cause modification of the photoresponsive material to produce a patterned distribution 208 of the modified material in skin region 202.

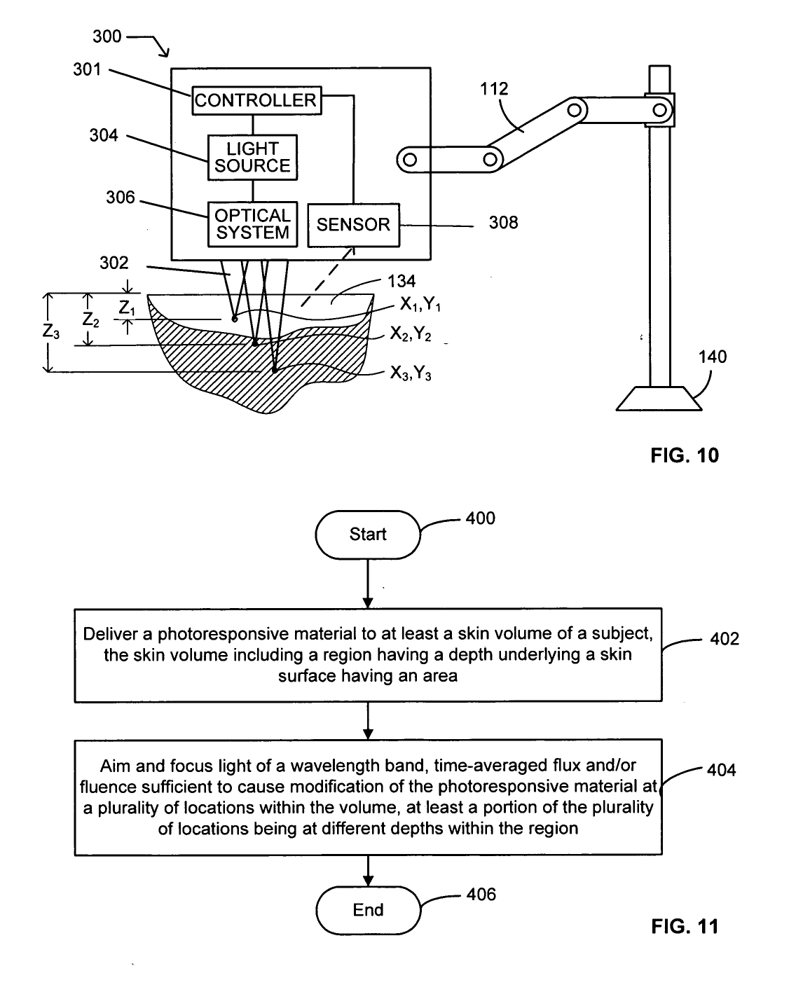

FIG. 10 provides a general illustration of a device 300 that may be used to produce a patterned distribution of light. Controller 301 controls the delivery of light 302 from light source 304 via optical system 306. Device 300 may be positioned by a mechanical linkage 112 supported by a base 140. Light 302 may be delivered at different x, y positions on the skin surface (e.g. x₁, y₁, x₂, y₂, x₃, and y₃ in FIG. 10), as well as at different depths or z positions (e.g.

z₁, z₂, and z₃ in FIG. 10) below the skin surface 134. Each location may be characterized by an x coordinate and y coordinate in an effectively planar portion of the skin region. Similarly, each location may be characterized by a z coordinate corresponding to the depth of the location below a surface of the skin region. In some applications, the z coordinate may be selected for each location such that a pattern is formed in the epidermis of the skin region. In other applications, the z coordinate may be selected for each location such that a pattern is formed in the dermis of the skin region, or even below the dermis. Also shown in FIG. 10 is sensor sub-system 308 for performing a sensing function to provide for feedback control of device 300. Sensor sub-system 308 may measure a parameter of skin surface 134, either prior to or subsequent to the application of the light (e.g., skin color, temperature, or conductance, distance of device 300 from skin surface 134, or one or more other parameters) for controlling some aspect of application of light by device 300.

A method as depicted in FIG. 11 may be used for forming a pattern in a skin volume. At step 402, a photoresponsive material is delivered to at least a skin volume of a subject, the skin volume including a region having a depth underlying a skin surface having an area. At step 404, light of a wavelength band, time-averaged flux and/or fluence sufficient to cause modification of the photoresponsive material may be aimed and focused at a plurality of locations within the volume, with at least a portion of the plurality of locations being at different depths within the region.

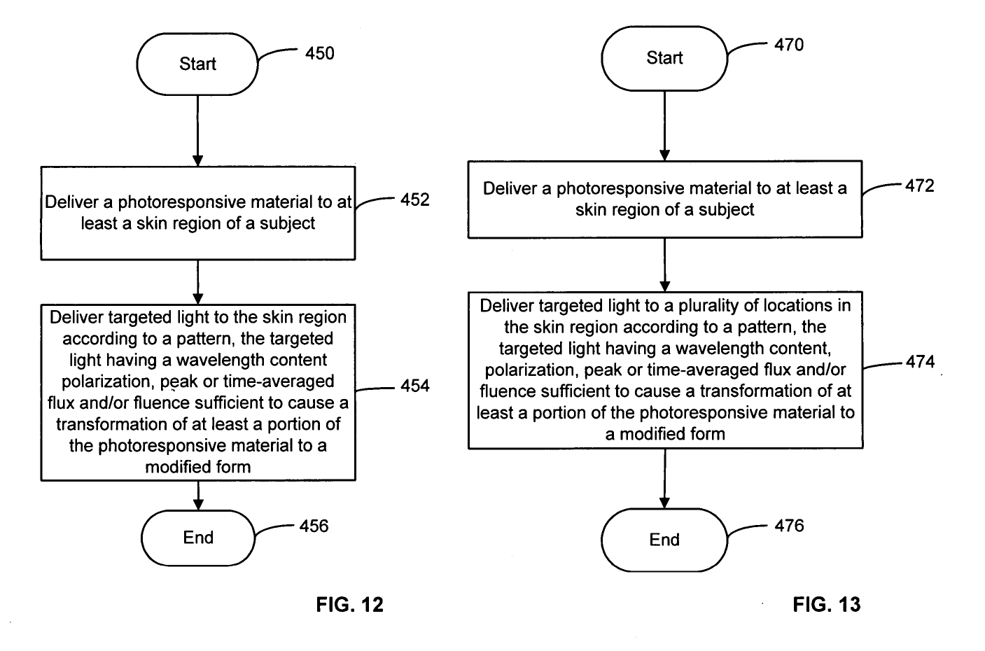

FIG. 12 depicts steps of a method of forming a patterned distribution of material in skin, including delivering a photoresponsive material to at least a skin region of a subject at step 452 and delivering targeted light to the skin region according to a pattern, the targeted light having a wavelength content, polarization, peak or time-averaged flux and/or fluence sufficient to cause a transformation of at least a portion of the photoresponsive material to a modified form, at step 454. FIG. 13 depicts a related method, which includes delivering a photoresponsive material to at least a skin region of a subject at step 472 and delivering targeted light to a plurality of locations in the skin region according to a pattern, the targeted light having a wavelength content, polarization, peak or time-averaged flux and/or fluence sufficient to cause a transformation of at least a portion of the photoresponsive material to a modified form, in step 474.

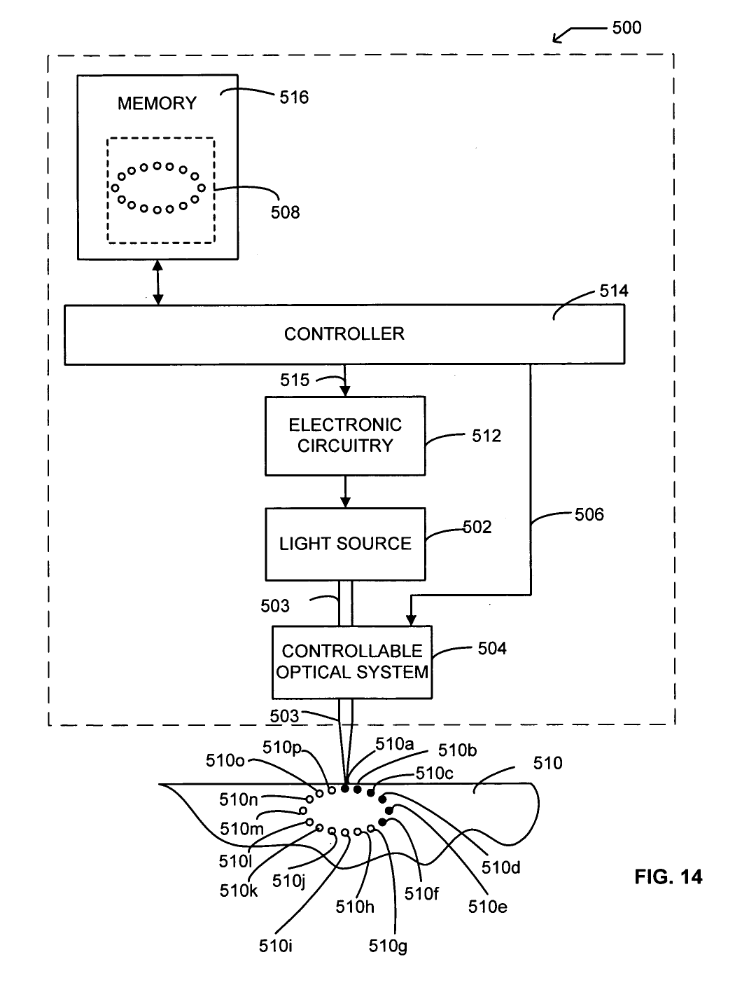

FIG. 14 is a block diagram of a system 500 for delivering patterned light. System 500 includes a light source 502 capable of producing light 503 of at least one defined wavelength band, and a controllable optical system 504. Controllable optical system 504 is configured to receive control signal 506 generated according to a pattern 508, and responsive to the control signal 506 to aim and focus light 503 from the light source 502 onto one or more selected skin locations of the plurality of skin locations 510a-510p according to pattern 508. Pattern 508 may represent a desired distribution of a material to a plurality of locations in or on skin region 510. System 500 may also include electronic circuitry 512 configured to limit the peak flux or fluence of light 503 produced by the light source 502 to levels that are non-damaging or not significantly damaging to skin. Controller 514, which may be, for example, a microprocessor, may perform computations used to produce control signal 506 for controlling controllable optical system 504, and light source drive signal 515 for driving light production by light source 502. Electronic circuitry 512 may function to limit light source drive signal 515 to limit light generation to safe levels, as well as to provide feedback control capability via a sensor (not shown). In some embodiments, a system for delivering patterned light to skin may include a light source capable of producing light of at least one defined wavelength band, a controllable optical system, and electronic circuitry configured to limit the peak flux or fluence of light produced by the light source to levels that are non-damaging or not significantly damaging to skin. The controllable optical system may be configured to receive a control signal generated according to a pattern representing a desired distribution of a material to a plurality of locations in or on a skin region, and responsive to the control signal to aim and focus light from the light source onto one or more selected skin locations of the plurality of skin locations according to the pattern. The system for delivering patterned light may also include an imaging device adapted for imaging a skin region containing at least a portion of the plurality of skin locations. In some embodiments, the system may include a device driver including one or more of hardware, software, or firmware for generating the control signal based upon pattern data stored in a machine readable medium. In some embodiments, the controllable optical system may include one or more deflectors configured to aim light from the light source, and the position of at least one of the one or more reflectors may be controllable to aim light toward at least one of the plurality of skin locations. In some embodiments, the controllable optical system may include a positioner adapted to adjust the position of the light source. Deflectors may include mirror-type reflectors and surface-acoustic wave (SAW) Bragg-type deflectors, as well as electrically-steered refractive elements. In some embodiments, feedback control of patterning action may be provided.

Patterned light may be delivered in the form of discrete pulses applied at multiple locations, as depicted in FIG. 14. Patterned light may also be delivered by sweeping a focused beam of light across a skin surface in a continuous pattern, for example, as depicted in FIG. 15. A beam may be moved across the skin surface with the use of a scanning mirror or functionally-equivalent optical systems of other types, the design and use of which is well known to those of skill in the art. Patterned light may also be delivered in some combination of continuous and discrete light; for example, a beam may be swept across the skin surface to form contiguous portions of a pattern, but turned on and off (e.g., by either mechanical or electrical means, or combinations thereof) as the beam is moved to non-contiguous portions of the pattern.

FIG. 15 depicts a system 600 including a controllable positioning system 602 that may be used to move a beam of light 604 over a skin surface 606 and to adjust the positioning of light from the light source on a skin region. System 600 may include a controllable optical system 608 that includes one or more deflectors 610 configured to aim light 604, from the light source 612. The position of at least one deflector 610 may be controllable to aim light 604 toward at least one of the plurality of skin locations. Controllable optical system 608 may include a positioner adapted to adjust the position of light source 612. Light source 612 may be capable of producing light 604 of at least one defined wavelength band. System 600 may also include memory 614 capable of storing a pattern 616 in machine-readable form representing a plurality of locations within a skin region to which light 604 from light source 612 is to be directed. In some embodiments, system 600 may include one or more optical components capable of focusing light 604 from the light source 612 at a specific depth within a skin region 12 in response to a control signal 618, controller 620 configured to generate control signal 618 for driving controllable positioning system 602 to direct light onto a plurality of skin locations according to pattern 616 stored in memory 614. Controller 620 may be configured to generate a control signal from driving one or more optical components to adjust the focusing of light 604 at different depths and at different skin locations according to pattern 616, and may be informed in at least one of its operations by at least one sensor 624 of skin condition. Deflectors 610 may be controllable deflectors configured to aim light 604 from light source 612, wherein the position of at least one of the one or more deflectors 610 is controllable to aim light toward any of the plurality of skin locations. Controller 620 may include one or more of hardware, software, and firmware. In some embodiments, controller 620 may include a microprocessor. In some embodiments, system 600 may include an imaging device, which may be for example, a CCD camera.

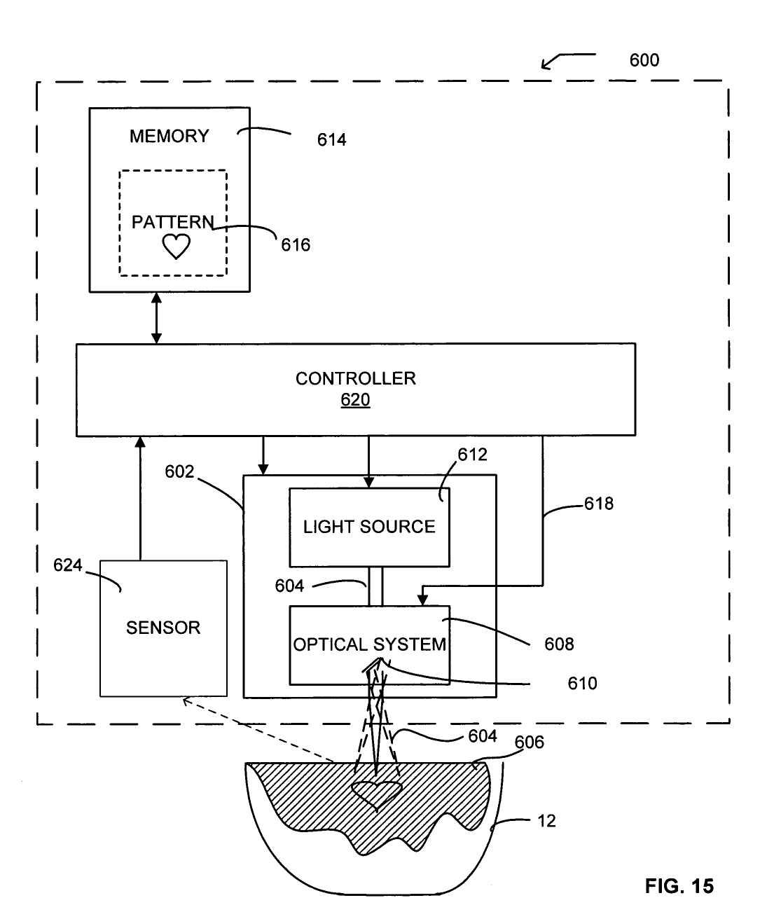

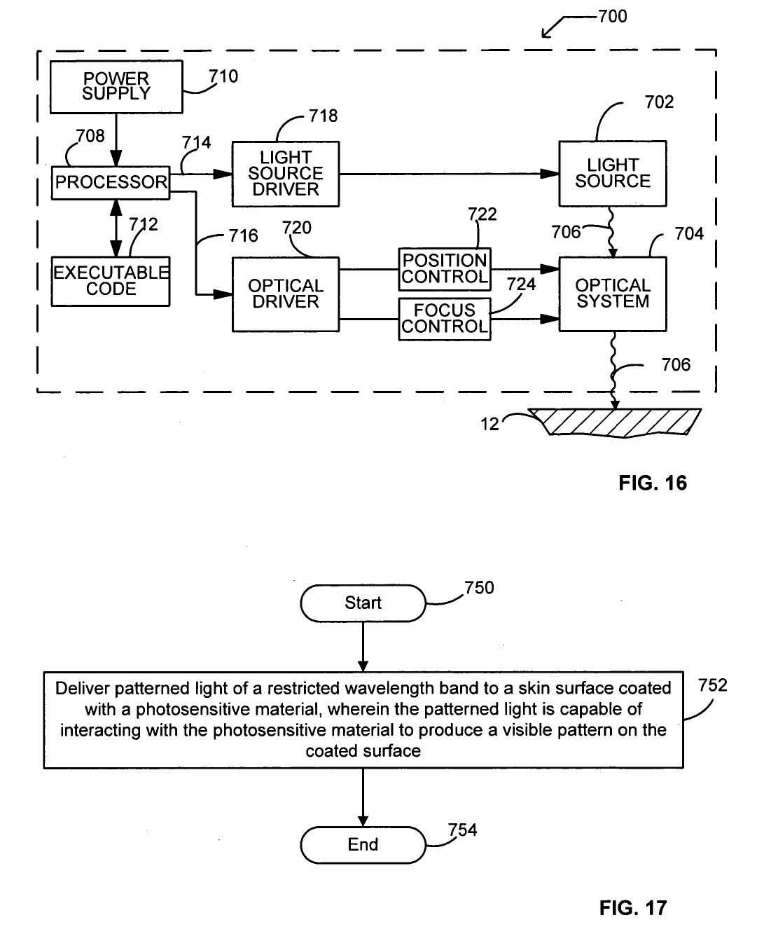

FIG. 16 is a block diagram of different aspects of a system 700 for delivering patterned light to a skin region 12. System 700 may include light source 702 and optical system 704, which directs and focuses light 706 from light source 702. Overall system operation may be controlled by processor 708, which may be, for example, a microprocessor, powered by power supply 710. Processor 708 may execute commands from executable code 712 to generate signals 714 and 716, which are sent to light source driver 718 and optical driver 720, respectively. Light source driver 718, which may include hardware, software, firmware, or a combination thereof, drives operation of light source 702. Optical driver 720, which also may include hardware, software, firmware, or a combination thereof, drives operation of optical system 704, via position control module 722 and focus control module 724. System 700 may be used to deliver targeted light to a plurality of locations under software control and/or under microprocessor control, and may include feedback control.

FIG. 17 outlines a method that includes delivering patterned light of a restricted wavelength band to a skin surface coated with a photosensitive material, wherein the patterned light is capable of interacting with the photosensitive material to produce a visible pattern on the coated surface, as shown at step 752 of the flow diagram. The photosensitive material may be applied to the surface. Light may be delivered to different locations in sequence, in either discrete or continuous fashion. Patterned light as used in certain embodiments may be produced with the use of a controllable optical system that is controllable to focus the light source on at least two of a plurality of skin locations in sequence. In some embodiments, a controllable optical system may be used that is controllable to focus the light source on at least two of a plurality of skin locations simultaneously.

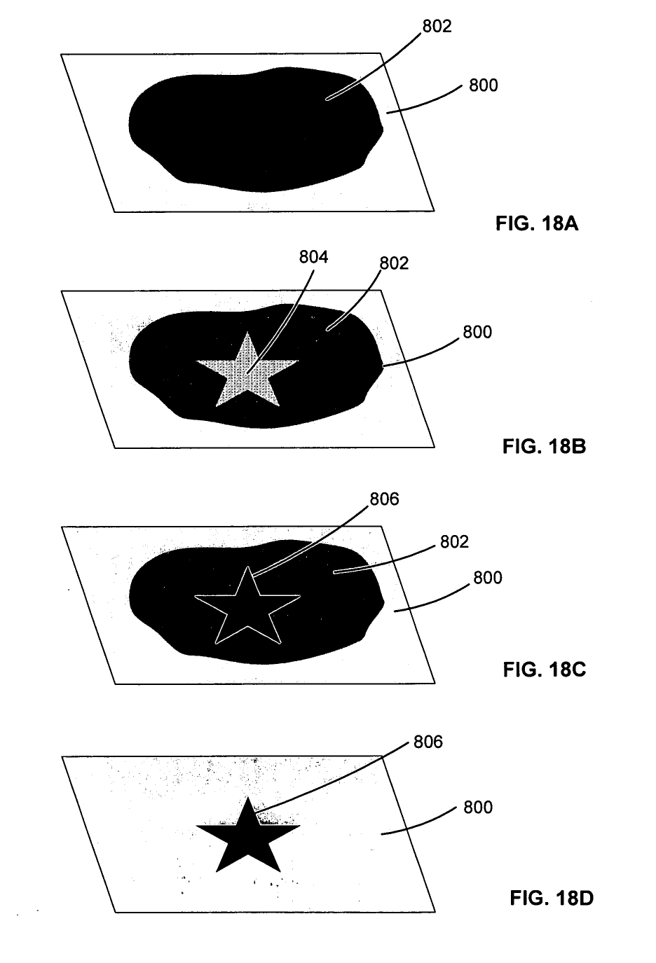

In some embodiments, light may be delivered to all parts of a pattern simultaneously. FIG. 18A illustrates a skin region 800 with a treated region 802 that contains a photoresponsive material. As described previously, photoresponsive material may be delivered to region 802 topically, by injection, regionally, or systemically. In step 18B, patterned light is delivered to area 804 in region 802 through the use of a stencil or mask or other methods as described herein below. Patterned light causes a reaction or transformation of at least a portion of photoresponsive material in area 804, to produce a pattern 806 of modified material as shown in FIG. 18C. In some embodiments, an additional step may be carried out to remove unmodified photoresponsive material from skin region 800, so that only pattern 806 remains in skin region 800, as depicted in FIG. 18D.

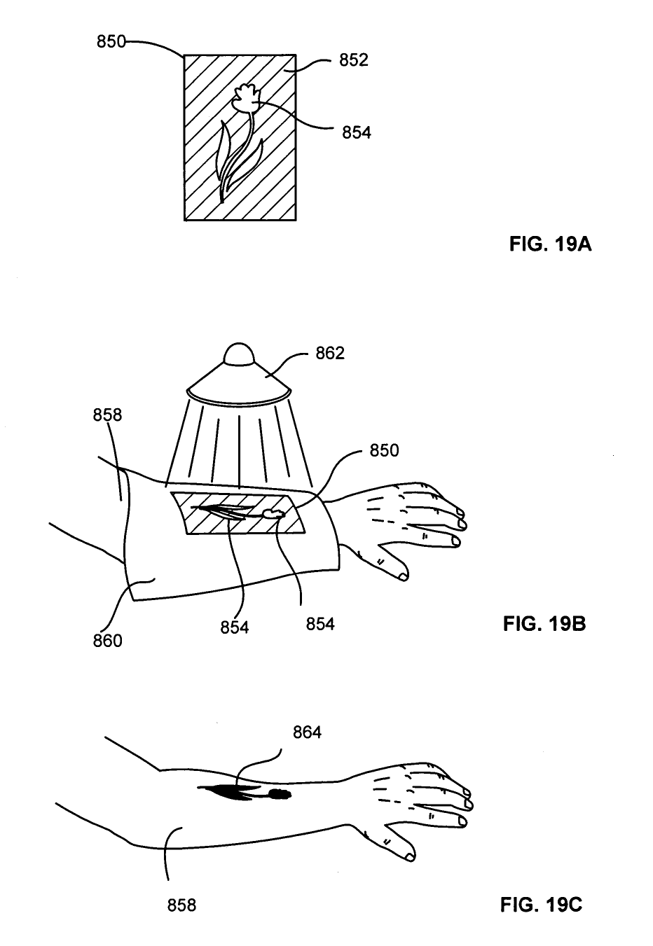

Several methods may be used to expose a treated skin region to patterned light. As shown in FIGS. 19A-19C, a mask (or stencil) 850 may be placed on the skin surface to block exposure of the skin surface to light except in the areas that are to be patterned. FIG. 19A depicts a mask 850 having an opaque portion 852 and a light transmitting portion 854. Mask 850 may be placed over a skin region that contains a photoresponsive material. In the example of FIG. 19B, the skin region is a portion of the arm 858 of a subject. A drape 860 may be used to extend the covered area of arm 858; various functionally-equivalent configurations may be devised by a practitioner of skill in the relevant art. Light from light source 862 may cover all of light transmitting portion 854 of mask 850, as depicted in FIG. 19B. In some alternative embodiments, light from a light source may cover a portion of a light transmitting portion of a mask, and the light source may be moved to one or more additional regions in order to expose all of the skin region exposed by the light transmitting portion of the mask. Light source 862 may be removed or turned off following exposure to light for a period of time sufficient to produce a desired modification of the photoresponsive material, and mask 850 and drape 860 (if used) removed. As shown in FIG. 19C, arm 858 of the subject bears a patterned distribution 864 of modified photoresponsive material that corresponds to the light transmitting regions 854 of mask 850.

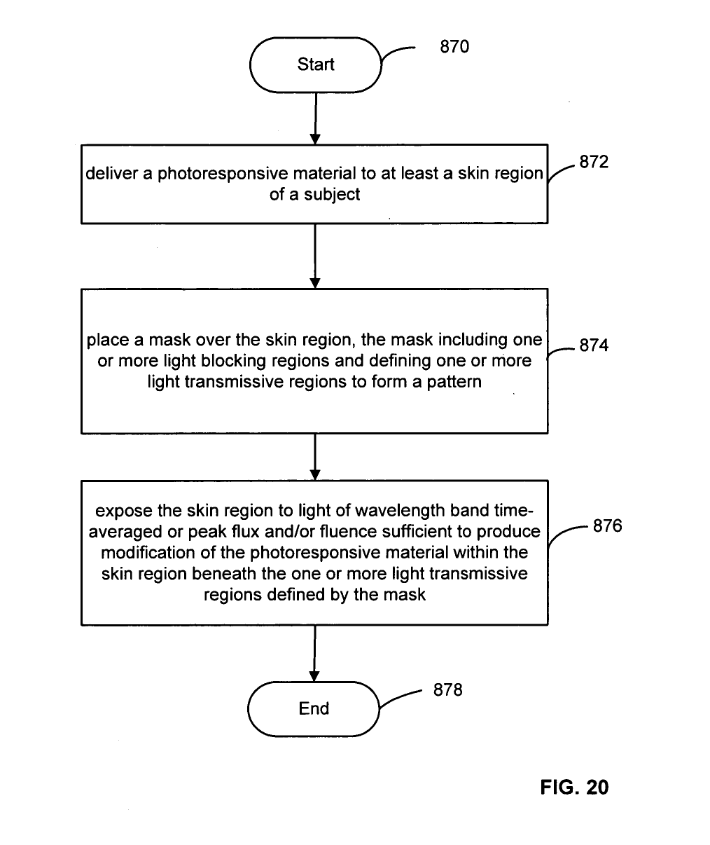

The method illustrated in FIGS. 19A-19C is summarized in FIG. 20. At step 872, a photoresponsive material is delivered to at least a skin region of a subject. At step 874, a mask is placed over the skin region, the mask including one or more light blocking regions and defining one or more light transmissive regions to form a pattern. At step 876, the skin region is exposed to light of wavelength band, time-averaged or peak flux and/or fluence sufficient to produce sufficient modification of the photoresponsive material within the skin region beneath the one or more light transmissive regions defined by the mask. Delivering a photoresponsive material may include delivering a photoresponsive material that is converted from an active form to an inactive form by exposure to light. Alternatively, delivering a photoresponsive material may include delivering a photoresponsive material that is converted from an inactive form to an active form by exposure to light. In further embodiments, the method may also include reversing the photo-reaction by exposing the skin region to light of a wavelength band, time-averaged or peak flux and/or fluence sufficient to reverse the reaction. Photo-reactions that may operate in a first direction at a first wavelength band, time-averaged or peak flux and/or fluence, and which may be reversed at a second wavelength band, time-averaged flux and/or fluence include, for example cross-linking of PEG-cinnamylidine acetate as described in U.S. Pat. No. 5,990,193, and reactions of various aromatic diazo dyes, as described in U.S. Pat. No. 5,998,588, both of which are incorporated herein by reference in their entirety.

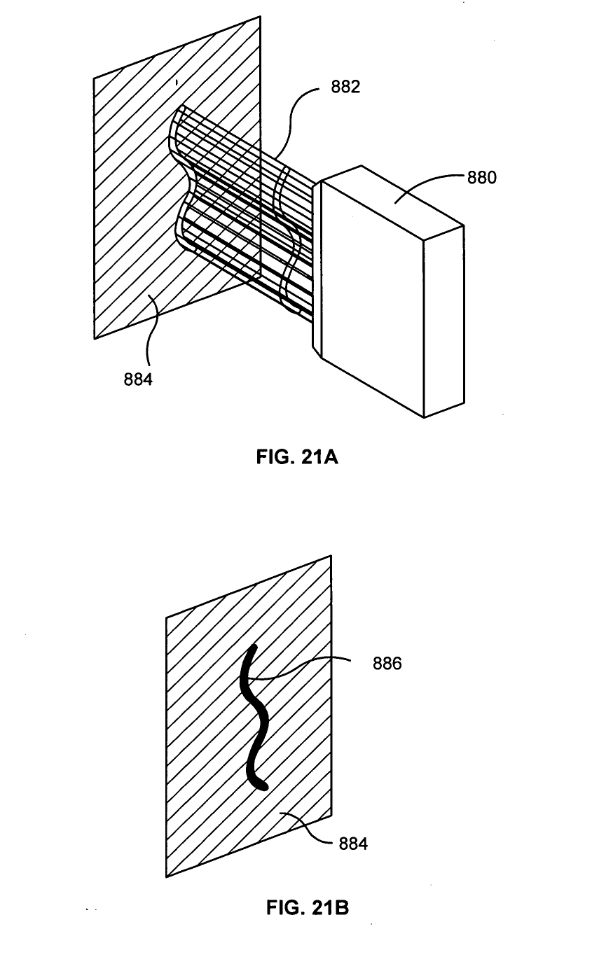

An alternative method of delivering patterned light is depicted in FIGS. 21A and 21B. FIG. 21A depicts a light source 880 that produces patterned light 882. This may be accomplished by placing a mask over a single light source of sufficient size and capable of generating substantially collimated light, or by placing multiple smaller light sources, also capable of producing relatively parallel light, in a suitable arrangement. Patterned light 882 from light source 880 may then be delivered to a treated surface 884. In the example of FIG. 21A, treated surface 884 need not be masked, because the light is patterned, although in some embodiments patterned light may be used in combination with a mask or stencil. FIG. 21B illustrates pattern 886 that has been formed by modification of photoresponsive material in or on treated surface 884 by exposure to patterned light 882.

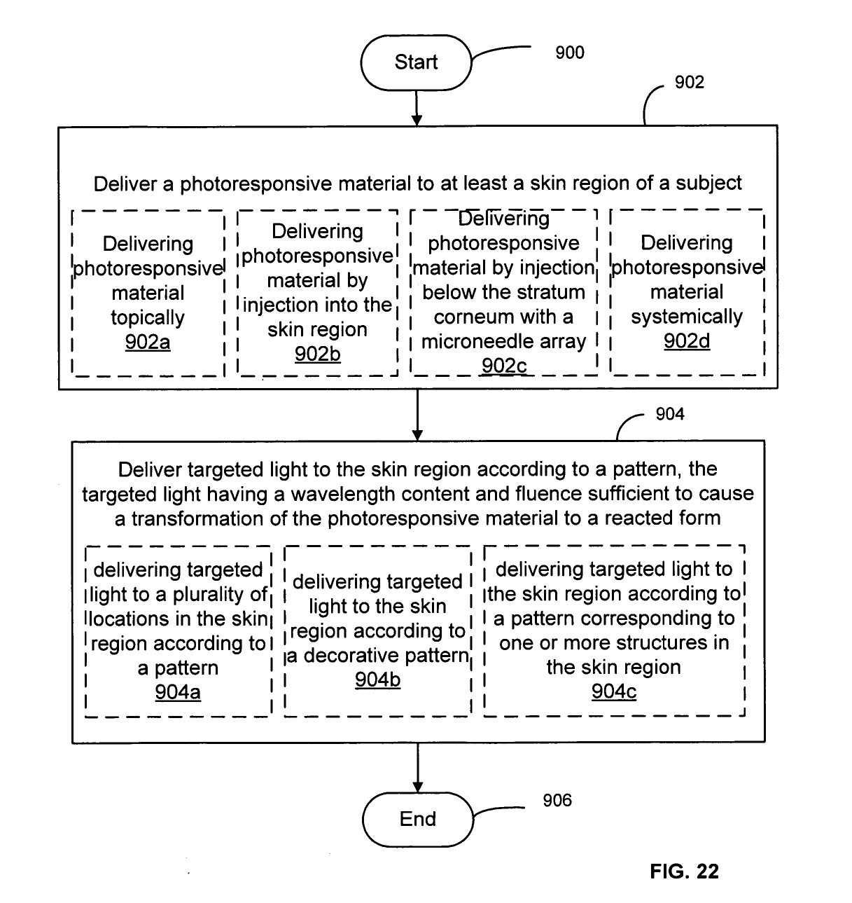

As illustrated in FIG. 22, various methods of delivering photoresponsive material to a skin region may be combined with various methods of delivering targeted light to a skin region to produce a number of related embodiments. Delivering photoresponsive material to at least a skin region of a subject, at step 902, may be further characterized as delivering photoresponsive material topically (step 902a), delivering photoresponsive material by injection in the skin region (902b) by delivering photoresponsive material by injection below the stratum corneum with a microneedle array (902c), or delivering the photoresponsive material systemically (902d). Delivering targeted light to the skin region according to a pattern, as at step 904, may be performed by a number of approaches, including delivering targeted light to a plurality of locations in the skin region according to a pattern (904a), delivering targeted light to the skin region according to a decorative pattern (step 904b) or delivering targeted light to the skin region according to a pattern corresponding to one or more structures in the skin region (step 904c). Methods including step 904c may also include a step of detecting one or more features in the skin region. The target light may have a wavelength content, time-averaged or peak flux, and/or fluence sufficient to cause a transformation of the photoresponsive material to a modified form. Distinctly different optical effects may be realized by differing means of delivery, and these delivery means may be employed at the same or differing times or process/patterning steps in a sequence thereof.

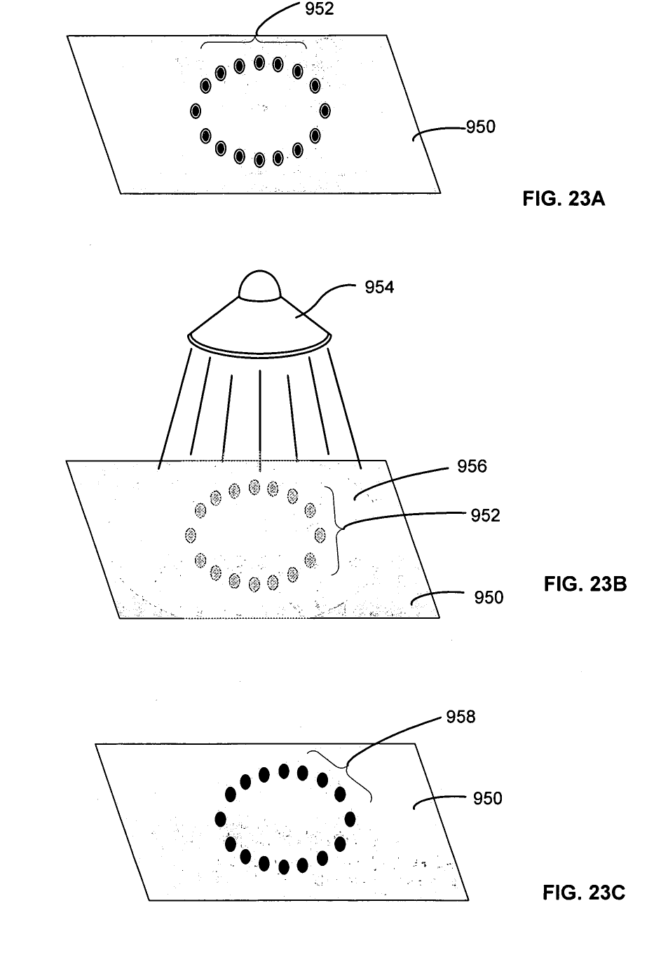

In some embodiments, a photoresponsive material may be introduced into a skin region in a patterned distribution, and light delivered to the skin in a relatively non-targeted fashion in order to cause transformation of at least a portion of the photoresponsive material to a modified form. This approach is illustrated in FIGS. 23A-23C. A photoresponsive material may be delivered topically in a pattern by various methods, including painting, printing (e.g., ink-jet or wire-jet printing), and stenciling, for example. Photoresponsive material may be delivered into the skin, below the skin surface, by injection with one or multiple needles (e.g. tattoo needles, micro-needle array, hypodermic needle) or by a pressure jet.

FIG. 23A illustrates a skin region 950 including a patterned distribution of photoresponsive material 952. In FIG. 23B, light source 954 is used to deliver light to a region 956 which includes patterned distribution of photoresponsive material 952. Light source 954 delivers light in a relatively non-targeted fashion; any light distribution that covers patterned distribution of photoresponsive material 952 with light of sufficient peak or time-averaged intensity or fluence may be used. In some embodiments, light may be delivered in several stages or from several sources, e.g., by delivering light from two or more sources, or from the same source at two different times, such that each individual delivery of light covers only a part of the patterned distribution of photoresponsive material, but that together, the multiple deliveries of light cover the entire patterned distribution of photoresponsive material. In FIG. 23C, following modification of photoresponsive material due to light exposure, a patterned distribution of modified material 958 is present in skin region 950.

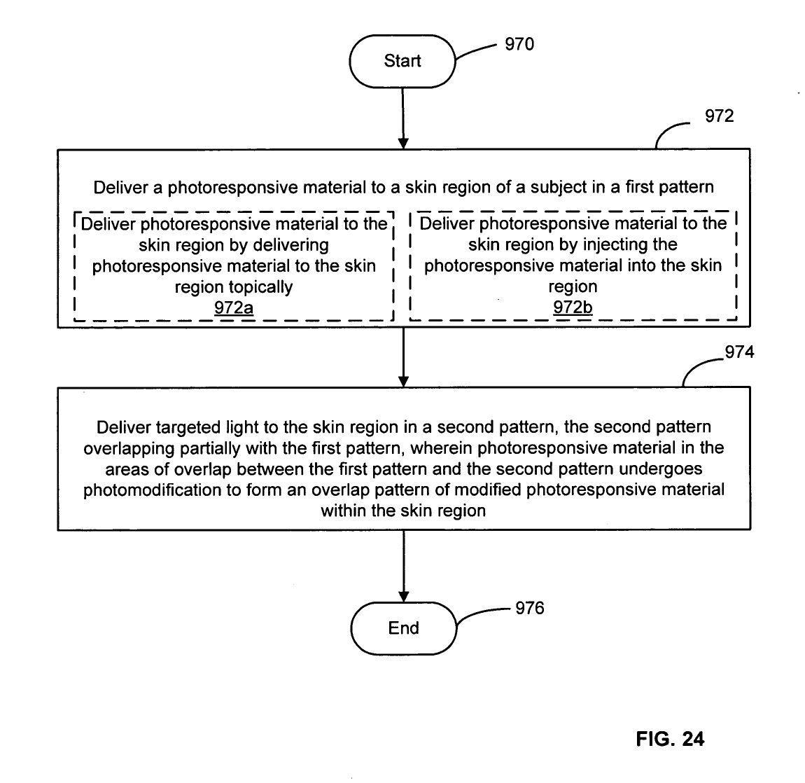

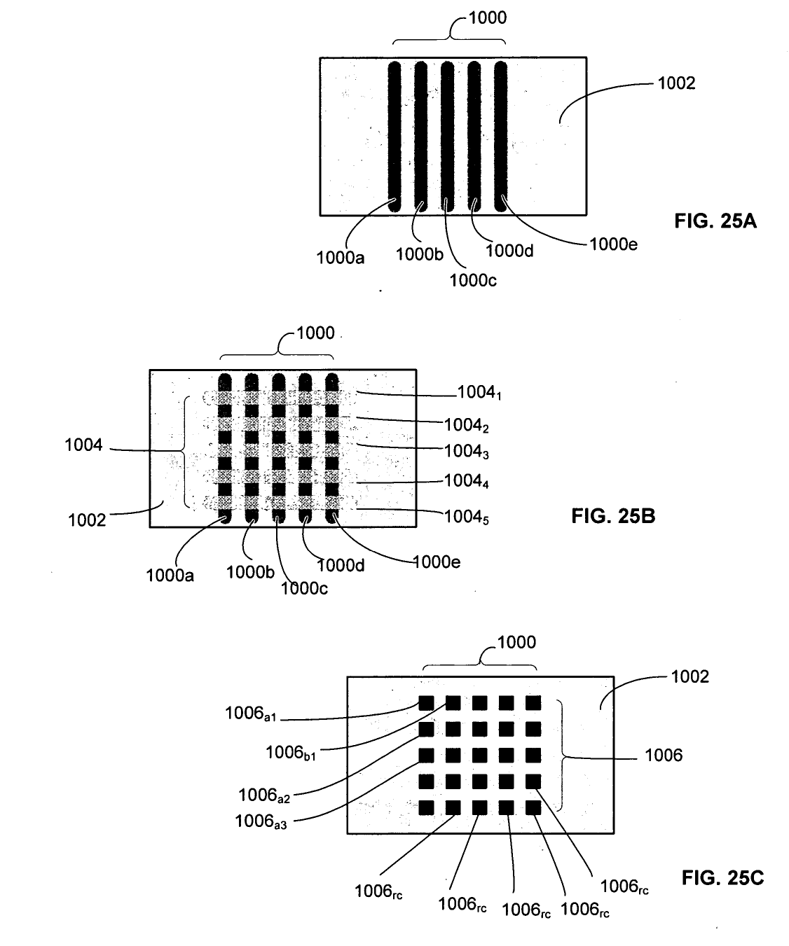

In some embodiments, both photoresponsive material and light may be delivered to the skin in a pattern. Patterned delivery of photoresponsive material and of light may be accomplished by any of the exemplary methods described herein above, for example. The patterns may be substantially similar and overlapping, in which case the distribution pattern of the modified form in or on the skin will be substantially the same as the distribution patterns of the unmodified form and the light. If the distribution pattern of the photoresponsive material and the distribution pattern of the light are partially overlapping, a patterned distribution of the modified form may be obtained that is defined by the shape and distribution of the regions of overlap between the distribution patterns of photoresponsive material and light. This approach is illustrated in FIG. 24 and FIGS. 25A-25C. At step 972 of FIG. 24, a photoresponsive material is delivered to a skin region of a subject in a first pattern. In one exemplary variant, 972a, photoresponsive material is delivered to the skin region topically. In another exemplary variant 972b, photoresponsive material is delivered to the skin region by injection (e.g., via a hypodermic needle, tattoo needle, microneedle array, pressure jet, etc.) At step 974, targeted light is delivered to the skin region in a second pattern, the second pattern overlapping partially with the first pattern. The photoresponsive material in the areas of overlap between the first pattern and the second pattern may undergo photomodification to form an overlap pattern of modified photoresponsive material within the skin region. The method is illustrated in graphic form in FIGS. 25A-25C. In FIG. 25A, a patterned distribution of photoresponsive material 1000 is formed in skin region 1002. In the present example, patterned distribution of photoresponsive material 1000 includes five lines of photoresponsive material 1000ₐ, 1000_{b}, 1000_{c}, 1000_{d}, and 1000ₑ. Such a patterned distribution may be formed by printing, injection, or other methods as described herein or as may be devised by one of skill in the art. In FIG. 25B, a patterned distribution of light 1004 is delivered to skin region 1002, overlapping patterned distribution of photoresponsive material 1000. Patterned distribution of light 1004 in this example includes five lines of light, 1004₁, 1004₂, 1004₃, 1004₄, and 1004₅, which may be formed by various methods as described previously. Following exposure to light, the photoresponsive material may react to form the patterned distribution 1006 of modified material in skin region 1002, as shown in FIG. 25C. Patterned distribution 1006 includes regions 1006_{rc}, where r=1 . . . 5 and c=a . . . e, formed by areas of overlap between patterned distribution of photoresponsive material 1000 and patterned distribution of light 1004.

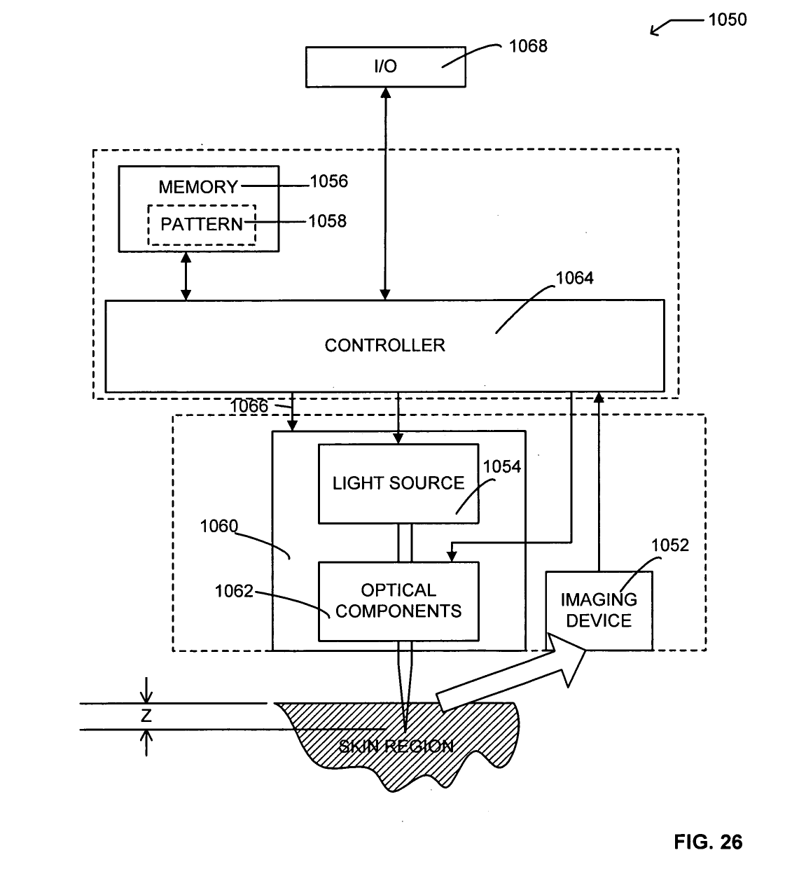

In some embodiments, it may be desirable to detect an image of a skin region in which a patterned distribution of a material is to be formed. For example, it may be desirable to detect a feature in a skin region that may be a treatment target, prior to delivery of a treatment in a targeted or aligned fashion. Or, it may be desirable to view an image of the skin region in order to determine placement of a decorative pattern in or on the skin region, e.g., aligned relative to a portion of a previously-emplaced pattern. FIG. 26 is a block diagram of a system 1050 that includes an imaging device 1052. System 1050 may include a light source 1054 capable of producing light of at least one defined wavelength band, memory 1056 capable of storing a pattern in machine-readable form representing a plurality of locations within a skin region to which light from the light source is to be directed and/or a pattern to be created, controllable positioning system 1060 configured to adjust the positioning of light from light source 1054 on a skin region, one or more optical components 1062 capable of focusing light from the light source 1054 at a specific depth within a skin region in response to a control signal, and controller 1064 configured to generate a control signal 1066 for driving controllable positioning system 1060 to direct light onto a plurality of skin locations according to the pattern 1058 stored in memory 1056. In some embodiments, controller 1064 may be configured to generate control signal 1066 for driving optical components 1062 to adjust the focusing of light at different depths and at different skin locations according to pattern 1058 stored in memory 1056. System 1050 may include additional sensing components or subsystems (not shown) for detection of at least one aspect or feature or portions of the skin or the pattern being formed on the skin. In some embodiments, controllable positioning system 1060 includes one or more controllable deflectors configured to aim light from light source 1054, wherein the position of at least one of the deflectors is controllable to aim light toward any of the plurality of skin locations. System 1050 may also include one or more I/O devices 1068 to provide for entry of control inputs by a user and for the presentation of information or data to the user. Various types of I/O devices are known or may be developed by those of skill in the arts of electronics and sensors for receipt and presentation of information and data in audio, visual, electronic, tactile, or other form, examples of which include scanners, touch screens, keyboards, mice, trackballs, buttons, dials, microphones, speakers, video displays, etc. Controller 1064 may include one or more of hardware, software, and firmware. In some embodiments, controller 1064 may include a microprocessor. System 1050 may include an imaging device, which may be, for example, a CCD camera, as well as a sensor sub-system that enables the feedback capabilities referenced above.

In various embodiments, the skin in or upon which a pattern is to be formed may be pre-treated in order to render it particularly amenable to the patterning process. For example, it may smoothed or ‘planarized’ (made locally ‘flat’) to control the optical characteristics of the skin before, during, or after the patterning process, or to render the patterning particularly adherent or durable, etc. Smoothing of the skin may be accomplished by various methods as are known in the art, e.g. abrasion, laser treatment, etc.

In various embodiments, examples of which are described herein, photoresponsive materials may be delivered to at least a skin region of a subject, and some or all of the photoresponsive material may be exposed to light to cause a reaction or conversion of the photoresponsive material. In some applications it may be desirable to remove one or both of modified and unmodified material from the subject's body. Unwanted material may be removed by processes normally occurring in the body, such as metabolism or excretion of the material, or by sloughing of skin containing the material. In some cases, materials may not be removed by naturally occurring processes, or may not be removed as quickly as is deemed desirable, and further treatment steps may be used to remove the materials from the body. In some embodiments, unmodified material may be removed, while modified material may be left in the skin region. In some embodiments, modified material may be removed from the skin region after a use period. Treatment to removed either modified or unmodified photoresponsive material, or both, may include phototreatment (e.g., photobleaching), chemical treatment (e.g., chemical bleaching, oxidizing, reducing, or application of at least one solvent), chemo-mechanical treatment (e.g., rinsing or scrubbing with a fluid which may include a surfactant), or treatment by exposure to at least one of heat, cold, pressure, vibration, electromagnetic fields, among others.

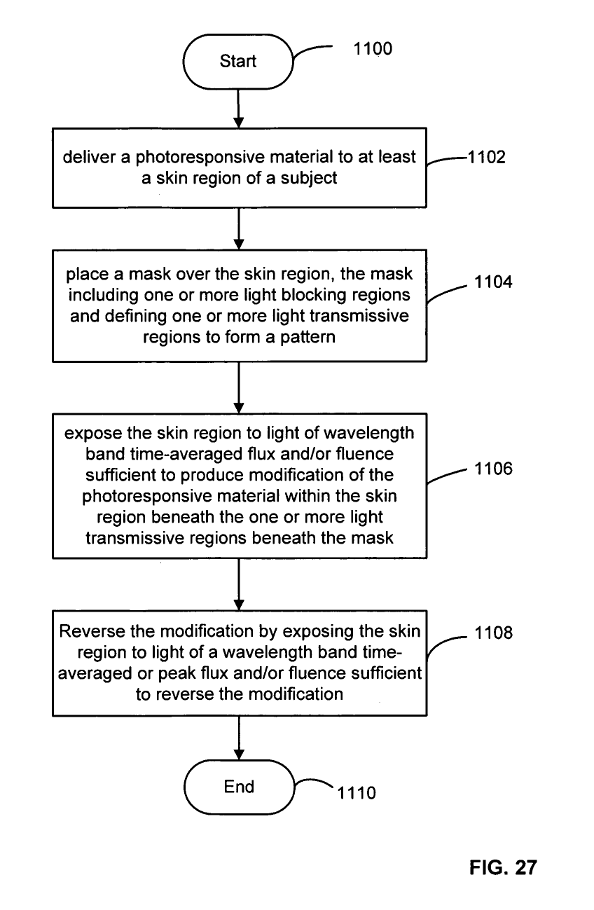

FIG. 27 depicts an exemplary sequence of method steps. At step 1102, a photoresponsive material is delivered to at least a skin region of a subject. At step 1104, a mask is placed over the skin region, the mask including one or more light blocking regions and defining one or more light transmissive regions to form a pattern. At step 1106, the skin region may be exposed to light of wavelength band, time-averaged flux and/or fluence sufficient to produce modification of the photoresponsive material within the skin region beneath the one or more light transmissive regions beneath the mask. Method steps 1102 through 1106 correspond to the method illustrated in FIGS. 19A-19C, for example. At step 1108, the modification is reversed by exposing the skin region to light of wavelength, time-averaged or peak flux and/or fluence sufficient to reverse the modification.

Various of the methods disclosed herein (for example, the method as outlined in FIG. 12), may include removal of the modified form of the photoresponsive material from the skin region over time. In some embodiments, the modified form may be removed from the skin region by metabolism. The modified form may be removed from the skin region through sloughing of dead skin cells and/or the continual shedding of epidermal outer layers, for example. In some embodiments, the modified form may be removed from the skin region after a treatment period. The method may include removing the modified form by a photo treatment, by a chemical treatment, or by a chemo-mechanical treatment.

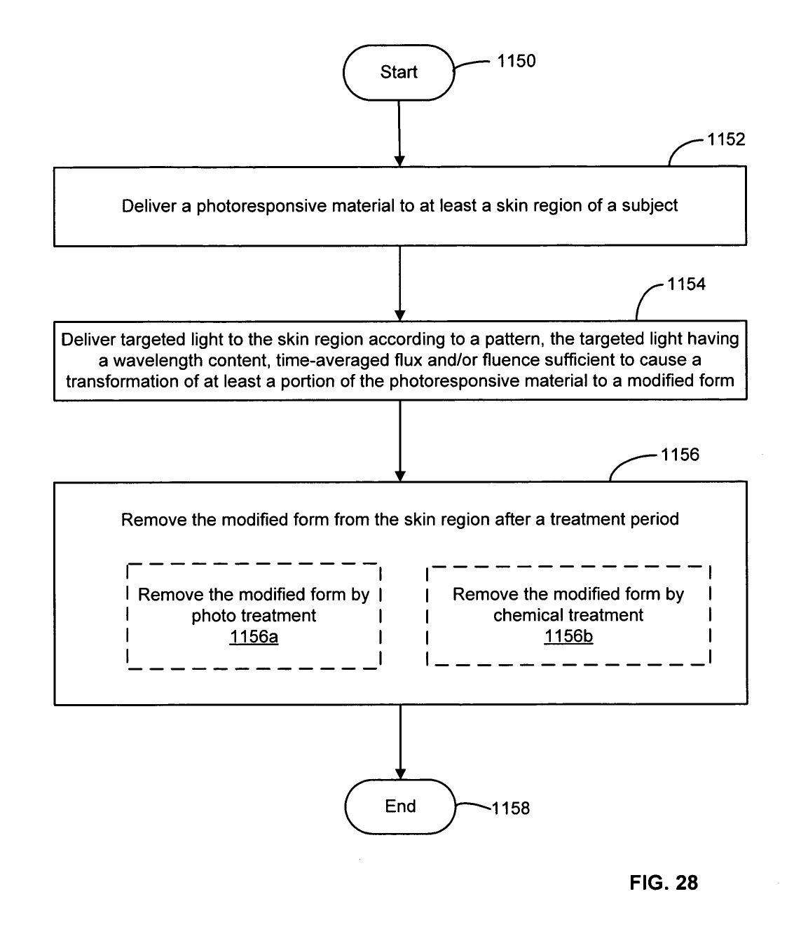

FIG. 28 depicts steps of a method that includes removing the modified form of the photoresponsive material from the skin region after a treatment period. At step 1152, a photoresponsive material is delivered to at least a skin region of a subject. At step 1154, targeted light is delivered to the skin region according to a pattern, the targeted light having a wavelength content, time-averaged flux and/or fluence sufficient to cause a transformation of at least a portion of the photoresponsive material to a modified form. At step 1156, the modified form is removed from the skin region after a treatment period. The modified form may be removed by photo treatment (step 1156a) or by chemical treatment (1156b), for example. The treatment period may be quite brief, producing only a transient presence of the modified material in the system, or may be of extended duration, of hours, days, weeks, months, or even years.

Examples of photoresponsive materials that may be used in various embodiments include, but are not limited to photodynamic therapy agents, photochromic dyes and pigments, photo-cross-linkable materials, photopolymerizable materials, and photodimerizable materials, luminides, materials subject to photolytic reaction, light reactive polymers that change in conformation, volume, binding activity, drug activity, and hydrogels of various types. Various exemplary photoresponsive materials are described in U.S. Pat. Nos. 6,602,975; 5,998,588; 6,555,663; 5,990,193; and 6,818,018, which are incorporated herein by reference in their entirety. Photoresponsive materials may be cosmetic materials having selected color or other appearance properties. Reaction undergone by photoresponsive materials may be a reversible transformation or an irreversible transformation. In some embodiments, the transformation may convert the photoresponsive material from an active to an inactive form. In other embodiments, the transformation may convert the photoresponsive material from an inactive to an active form. The transformation may include, for example, conversion of a photoresponsive material from a substantially colorless form to a colored form, or from a colored form to a substantially colorless form, or from a soluble form to an insoluble form or vice versa. Examples of photochromic dyes are listed in U.S. Pat. No. 6,602,975, which is incorporated herein by reference. In some embodiments, the transformation may include conversion of the photoresponsive material from a first color to a second color, or may modify the extent or manner in which it scatters or converts or processes light of a given waveband. The modified form may be under natural light in some embodiments, or visible under at least one component of human-visible spectral light. In some embodiments, the modified form may be visible under ultraviolet light. In some embodiments, the modified form may be fluorescent or phosphorescent material, and in some embodiments the modified form may be a pigment, a dye, a refracting, diffracting, polarizing or reflective material, a pharmaceutical compound, or a cosmetic material.

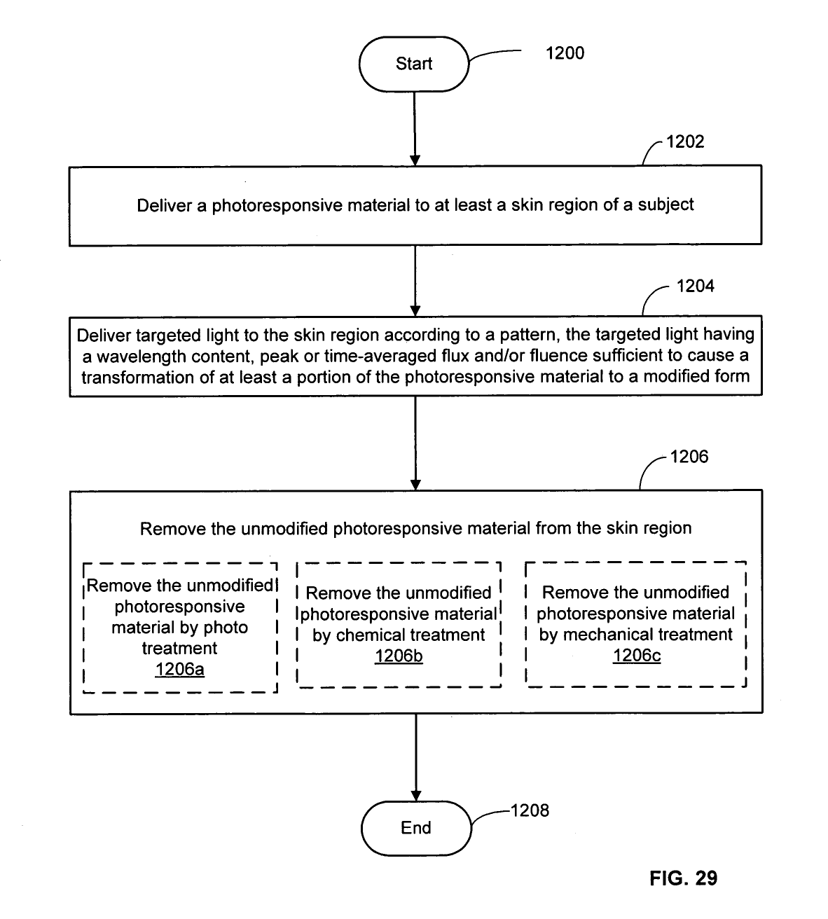

FIG. 29 depicts steps of a method that includes removing unmodified photoresponsive material from a skin region of a subject. At step 1202, a photoresponsive material is delivered to at least a skin region of a subject. At step 1204, targeted light is delivered to the skin region according to a pattern, the targeted light having a wavelength content, peak or time-averaged flux and/or fluence sufficient to cause a transformation of at least a portion of the photoresponsive material to a modified form. At step 1206, the unmodified photoresponsive material is removed from the skin region. The unmodified photoresponsive material may be removed by photo treatment, as shown in step 1206a, or by chemical treatment, as shown in step 1206b, or by mechanical treatment (e.g., scrubbing) at step 1206c or a combination of these.

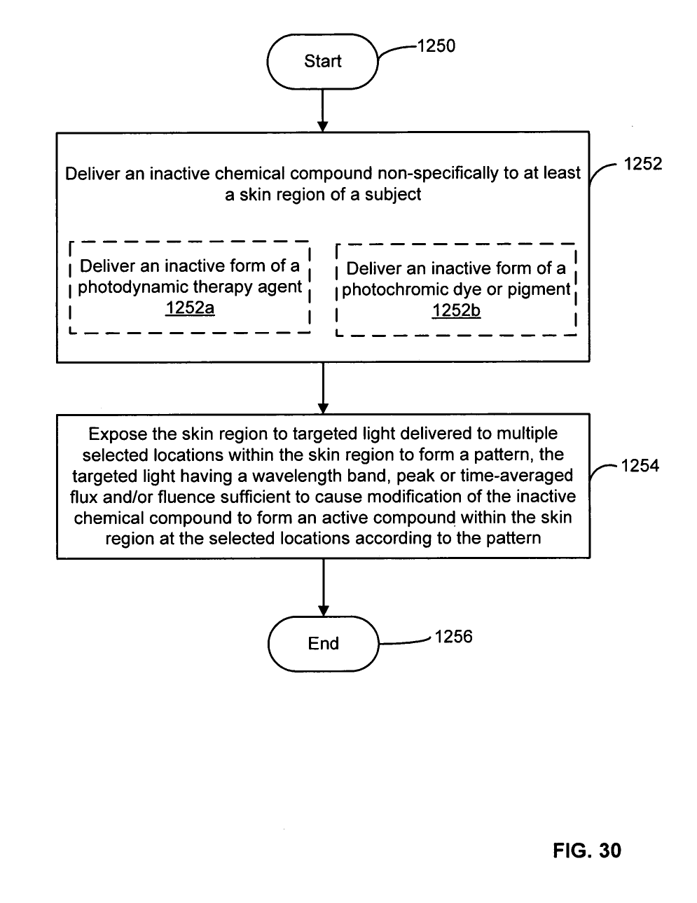

FIG. 30 illustrates a method of providing controlled delivery of an active compound to a skin region, which includes delivering an inactive chemical compound non-specifically to at least a skin region of a subject at step 1252 and exposing the skin region to targeted light delivered to multiple selected locations within the skin region to form a pattern at step 1254, the targeted light having a wavelength band, peak or time-averaged flux and/or fluence sufficient to cause modification of the inactive chemical compound to form an active compound within the skin region at the selected locations according to the pattern. As illustrated by steps 1252a and 1252b, respectively, delivering an inactive chemical compound may include delivering an inactive form of a photodynamic therapy agent or a photochromic dye or pigment. It is within the present inventive scope to deliver two-or-more materials in this manner, and to induce reactions between the two-or-more materials or between the two-or-more materials and ambient materials by the action of the incident light.

Systems for the delivery of light to skin, as described herein, may include various types of light sources. In general, suitable light sources must deliver light having wavelength content, fluxes and fluences sufficient to produce a particular effect in the photoresponsive material(s) that is (are) being exposed to the light. For example, in some embodiments, the light may have a wavelength content, peak or time-averaged flux and/or fluence sufficient to cause a photo-cross-linking reaction of the photoresponsive material. In other embodiments, the light may have wavelength content, peak or time-averaged flux and/or fluence sufficient to cause a photochromic reaction of the photoresponsive material. In still other embodiments, the light may have a wavelength content, peak or time-averaged flux and/or fluence sufficient to cause a photodimerization reaction or photolytic reaction of at least a portion of the photoresponsive material. Light sources suitable for use in various embodiments as described herein include lasers, laser diodes, as well as various non-coherent light sources. Light sources may include light emitting diodes. In some embodiments, light sources may emit light in an ultraviolet wavelength band. In some embodiments, light sources may emit light in a visible wavelength band, or in an infrared one. Broad-band (e.g., incandescent filament-based) light sources may be used in some embodiments.

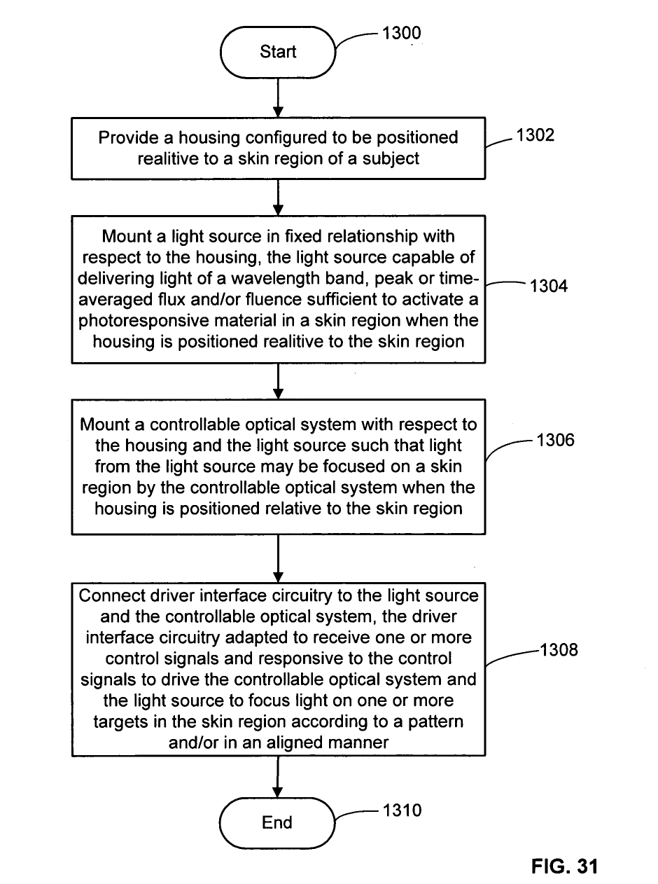

FIG. 31 depicts a method of manufacturing a targeted light delivery system. Step 1302 includes providing a housing configured to be positioned relative to a skin region of a subject. At step 1304, a light source is mounted in fixed relationship with respect to the housing, the light source capable of delivering light of a wavelength band, peak or time-averaged flux and/or fluence sufficient to activate a photoresponsive material in a skin region when the housing is positioned relative to the skin region. At step 1306, a controllable optical system is mounted with respect to the housing and the light source such that light from the light source may be focused on a skin region by the controllable optical system when the housing is positioned relative to the skin region. At step 1308, driver interface circuitry is connected to the light source and the controllable optical system, the driver interface circuitry adapted to receive one or more control signals and responsive to the control signals to drive the controllable optical system and the light source to focus light on one or more targets in the skin region according to a pattern and/or in an aligned manner. Alternatively, or in addition, the system may be driven in a manner responsive to feedback from the skin being patterned.

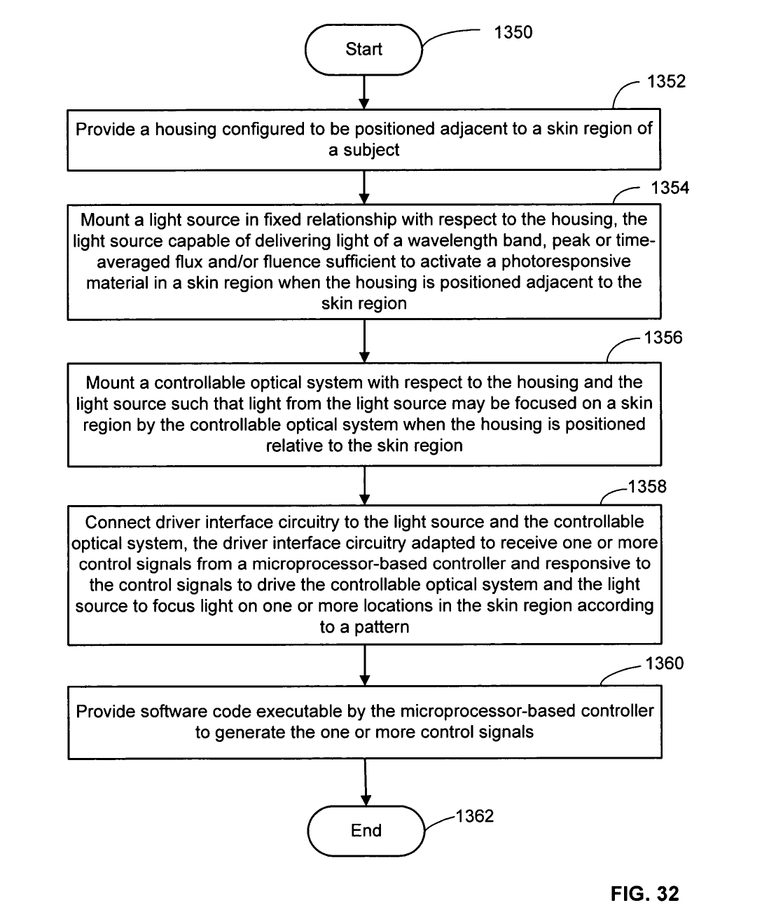

FIG. 32 depicts a method of manufacturing a device for delivering patterned light. At step 1352, a housing is provided that is configured to be positioned adjacent to a skin region of a subject. At step 1354, a light source is mounted in fixed relationship with respect to the housing, the light source capable of delivering light of a wavelength band, peak or time-averaged flux and/or fluence sufficient to activate a photoresponsive material in a skin region when the housing is positioned adjacent to the skin region. A controllable optical system is mounted with respect to the housing and the light source such that light from the light source may be focused on a skin region by the controllable optical system when the housing is positioned relative to the skin region at step 1356. At step 1358, driver interface circuitry is connected to the light source and the controllable optical system, the driver interface circuitry adapted to receive one or more control signals from a microprocessor-based controller and responsive to the control signals to drive the controllable optical system and the light source to focus light on one or more locations in the skin region according to a pattern. Alternatively, or in addition, control signals may be generated in response to feedback from the skin being patterned. At step 1360, software code is provided that is executable by the microprocessor-based controller to generate the one or more control signals. In some embodiments, the driver interface circuitry may be adapted to receive the one or more control signals from a microprocessor-based controller. In some embodiments, the method may include providing software code executable by the microprocessor-based controller to generate the one or more control signals.

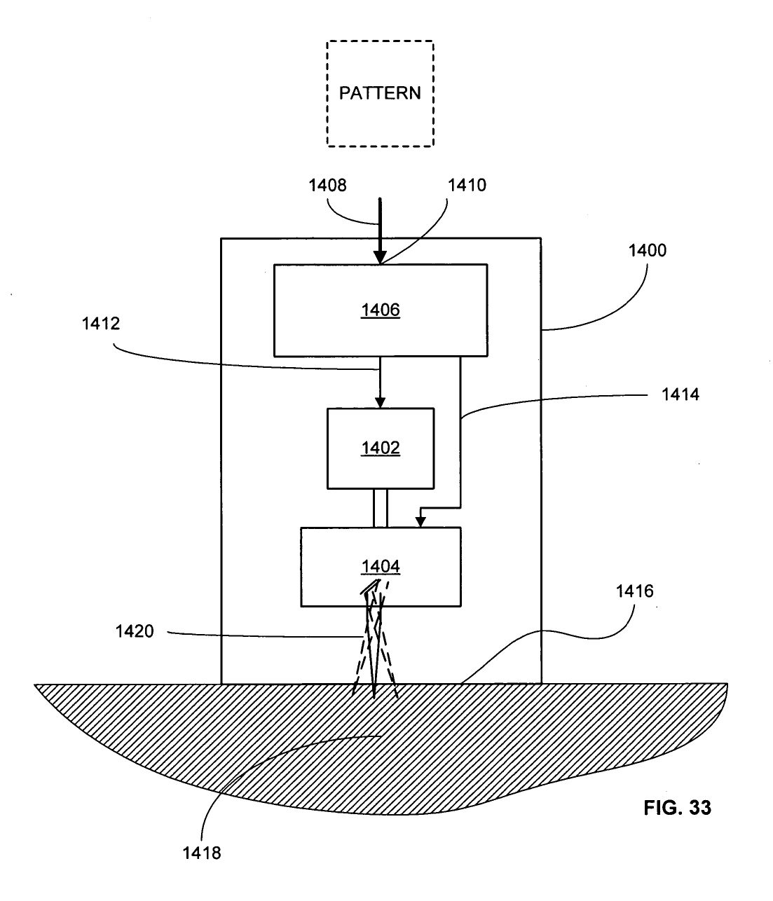

FIG. 33 depicts features of a device as described in connection with FIG. 32; included are housing 1400, light source 1402, controllable optical system 1404, and driver interface circuitry 1406. Driver interface circuitry receives at least one control signal 1408 on input 1410, and generates control signals 1412 and 1414 for driving light source 1402 and controllable optical system 1404, respectively. Portion 1416 of housing 1400 may be configured to be positioned adjacent a skin region 1418, so that light 1420 may be directed to skin region 1418 by controllable optical system 1404.

The methods, apparatuses, and approaches described herein may be modified and combined in a variety of ways analogous to those of photolithography of semiconductor (e.g., silicon) wafers. For example, masks or stencils may be used to form positive or negative patterns on, above or beneath the surface of skin. Additive and subtractive processing may be performed by appropriate combinations of steps. For example, multiple steps, each involving the use of a different stencil and a different depth of focus of light in the skin, may be used to form a patterned distribution of material that varies as a function of depth within the skin. As another example, a multi-step process may be used in which a material modified at a first step, for example by treatment at a first wavelength, may in turn influence (e.g. by causing, preventing, promoting, or inhibiting) a further reaction or modification of the same or a different material produced at a second step by treatment with a second wavelength. It will be appreciated that a wide variety of combinations of treatment steps may be devised to control formation of patterned distributions of material in skin. As with photolithography methods, as multiple steps involving patterned delivery of materials or light to the skin are used, it may be necessary to maintain alignment or registration of patterns delivered at each step, e.g. by controlling mask positioning or targeting of light or delivery of photoresponsive material. Methods of maintaining positioning, targeting, or alignment are known to those of skill in the art, and variations are considered to fall within the scope of the present invention.

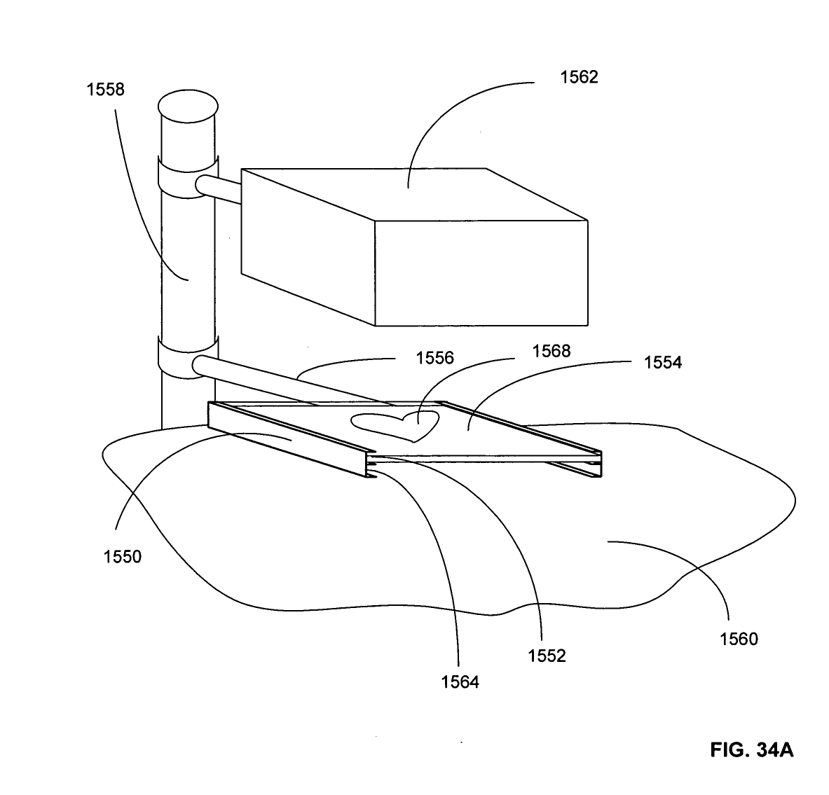

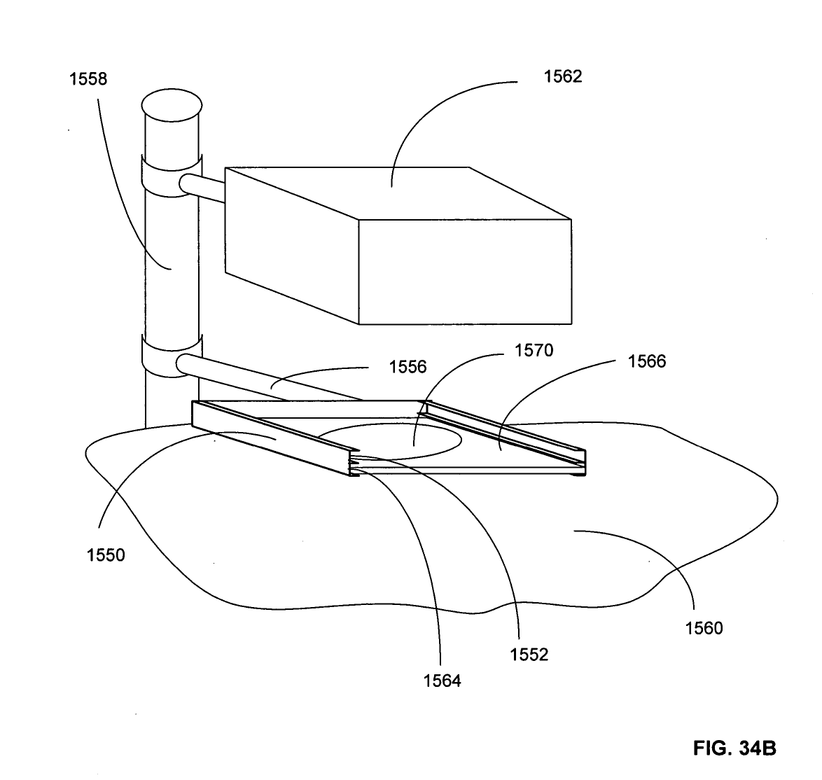

FIGS. 34A and 34B illustrate an embodiment of a system for positioning masks in proper alignment over a skin surface. In FIG. 34A, mounting 1550 includes first recess 1552 configured to receive first mask 1554. Mounting 1550 is supported by linkage 1556, which in the present exemplary embodiment is attached to post 1558. Post 1558 is positioned with respect to skin region 1560. Light delivery system 1562, which may include a light source, optical components, may also be positioned relative to skin region 1560 by means of post 1558. Mounting 1550 may include a second recess 1564, adapted to receive a mask. In an example of use of the embodiment depicted in FIGS. 34A and 34B, at a first step shown in FIG. 34A, light from light delivery system 1562 may be delivered to skin region 1560 through light transmissive region 1568 in first mask 1554. At a second step shown in FIG. 34B, light from light delivery system 1562 is delivered to skin region 1560 through light transmissive region 1570 in second mask 1566. In this example, first mask 1554 was removed from first recess 1552, and second mask 1566 was placed in second recess 1564, in registration with first mask 1554, but at a slightly different level. In some embodiments, second (or subsequent) masks may be placed in first recess 1552 rather that in a recess located at a different height relative to the skin region. The number of recesses and masks may be varied depending upon the intended application.

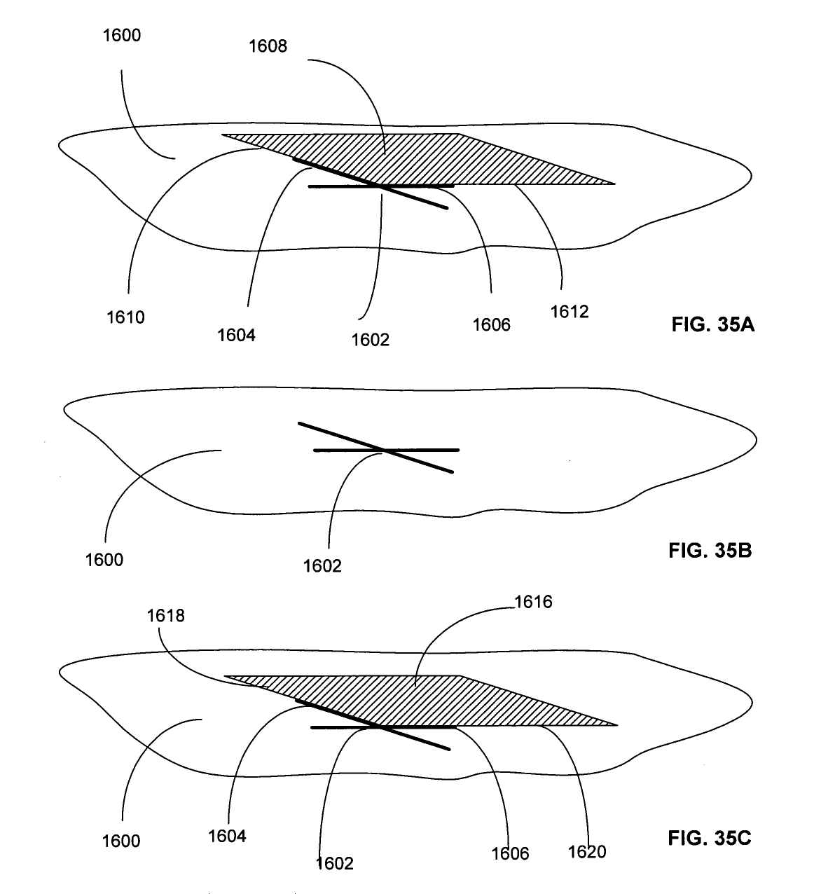

FIGS. 35A-35C illustrate the use of indicia marked on the skin for maintaining alignment of masks. In FIG. 35A, skin surface 1600 has cross-shaped marking 1602 made up of crossing lines 1604 and 1606. First mask 1608 is positioned on skin surface 1600 by aligning first edge 1610 with first line 1604 and second edge 1612 with second line 1606. After completion of a first step, utilizing first mask 1608, first mask 1608 is removed, as shown in FIG. 35B, and at FIG. 35C, second mask 1616 is positioned on skin surface 1600 by aligning first edge 1618 with first line 1604 and second edge 1620 with second line 1620.

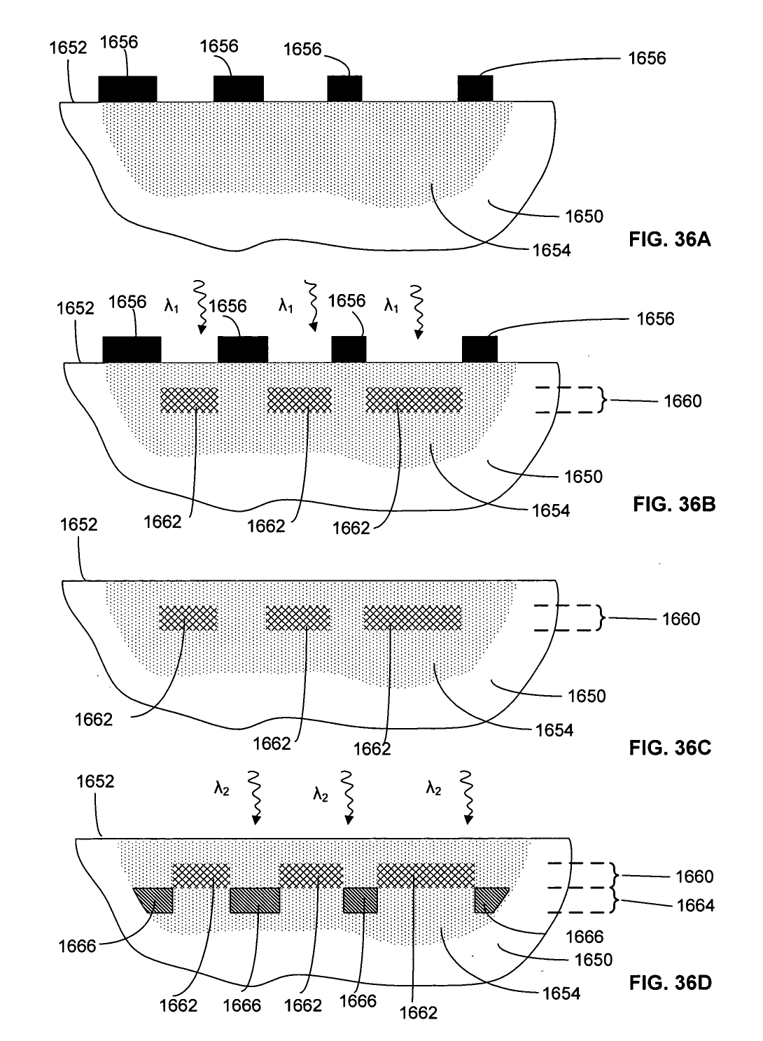

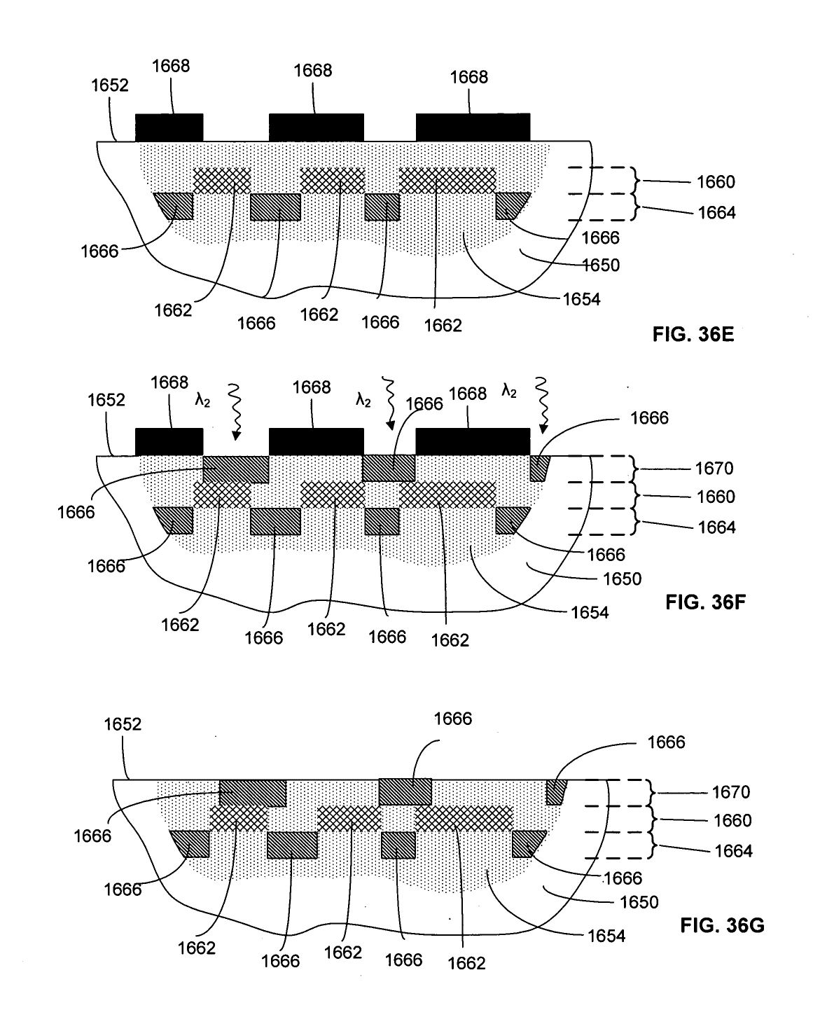

FIGS. 36A-36G provide an example of the use of multiple steps in the photopatterning of skin. It will be appreciated that this is only one of many possible combinations of previously described steps, and that various other combinations of such steps will be apparent to the practitioner of skill in the art. In FIG. 36A, a skin region 1650 is depicted in cross section, with the skin surface indicated by reference number 1652. Photoresponsive material 1654 may be present in at least a portion of skin region 1650. A mask 1656 may be placed on skin surface 1652. Light blocking regions of mask 1656 are indicated by black rectangles. The gaps between the light blocking regions of mask 1656 represent the light transmitting regions of mask 1656. As depicted in FIG. 36B, when light of wavelength λ₁ is focused at a first depth range 1660 in skin region 1650, photoresponsive material 1654 is modified to a first modified form 1662 at locations not blocked mask 1656. Mask 1656 is subsequently removed, leaving skin region 1650 containing first modified form 1662 at selected regions, as depicted in FIG. 36C. As depicted in FIG. 36D, when light of wavelength λ₂ is focused at a second depth range 1664 in skin region 1650, photoresponsive material 1654 is modified to a second modified form 1666 at locations not blocked by first modified form 1662. For example, first modified form 1662 may function to absorb, reflect, or otherwise modify the effect of light of wavelength λ₂. Second modified form 1666 is thus formed at multiple locations within second depth range 1664. In FIG. 36E, a second mask 1668 (including light blocking portions 1668 and light transmissive regions between the light blocking portions) is placed on skin surface 1652. Next, as depicted in FIG. 36F, light of wavelength λ₂ is focused at a third depth range 1670 in skin region 1650, photoresponsive material 1654 is modified to a second modified form 1666 at locations in third depth range 1670 not blocked by second mask 1668. Finally, as shown in FIG. 36G, the second mask may be removed, leaving skin region 1650 patterned with second modified form 1666 in second and third depth ranges 1664 and 1670, and patterned with first modified form 1662 at first depth range 1660. Depending upon the nature of first modified form 1662, it may be left in place in skin region 1650 or removed by various methods. Similarly, photoresponsive material 1654 may be left in skin region 1650, or removed by naturally occurring processes or by a specifically involved removal process (e.g., treatment with light, a chemical, etc.).

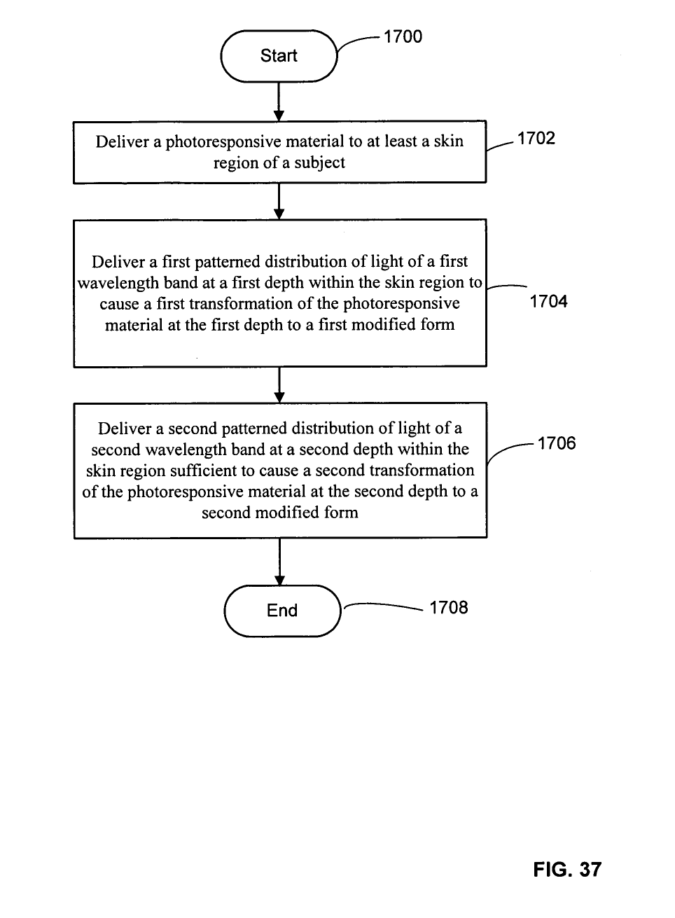

As outlined above and detailed in FIG. 37, a method of forming a patterned distribution of a material in or on skin may include delivering a photoresponsive material to at least a skin region of a subject at step 1702, delivering a first patterned distribution of light of a first wavelength band at a first depth within the skin region to cause a first transformation of the photoresponsive material at the first depth to a first modified form at step 1704, and delivering a second patterned distribution of light of a second wavelength band at a second depth within the skin region sufficient to cause a second transformation of the photoresponsive material at the second depth to a second modified form at step 1706.

A variety of parameters may be varied during the practice of the invention, in various combinations. In some embodiments, the first depth may be the same as the second depth. In other embodiments, the first depth may be different than the second depth. In some embodiments, the first wavelength may be the same as the second wavelength, while in others the first wavelength may be different than the second wavelength. The first patterned distribution of light may produce a first transformation of the photoresponsive material at the first depth, and the second patterned distribution of light may produce a first transformation of the photoresponsive material at the second depth. The first transformation of the photoresponsive material may include a conversion of the photoresponsive material from a first state to a second state, while the second transformation of the photoresponsive material may include a conversion of the photoresponsive material from a second state to a third state. In some cases, the first state may be equivalent to the third state, while in others the first state may be different from the third state. In some embodiments, the photoresponsive material may include two or more components, so that the first transformation of the photoresponsive material includes a modification of a first component of the photoresponsive material and the second transformation of the photoresponsive material includes a modification of a second component of the photoresponsive material. Components of photoresponsive materials may be transformed to produce modification of structural properties of the photoresponsive material or modification of light-modulating properties of the photoresponsive material. Modification of structural and light-modulating properties may be produced during separate method steps, in any order or simultaneously.

Delivery of photoresponsive material to the skin during multi-step methods may be performed in the same ways as in single-step methods. In some embodiments, photoresponsive material may be delivered to at least a skin region of a subject topically, for example in the form of an aerosol, cream, emulsion, gel, liquid, fluid, gas, vapor, lotion, patch, powder, or combination thereof. In some embodiments, photoresponsive material may be delivered to at least a skin region of a subject by injecting the photoresponsive material into the skin region. Photoresponsive material may be delivered to at least a skin region of a subject by injecting the photoresponsive material below the stratum corneum of the skin region with the use of a microneedle array. In other alternative embodiments, photoresponsive material may be delivered to at least a skin region of a subject by delivering the photoresponsive material to the subject systemically, which may be performed, for example, by delivering the photoresponsive material to the subject orally in an ingestible formulation.

The first and second transformations may be the same type of transformation, or they may be different types of transformations. In some embodiments, one transformation may reverse the other transformation. In some embodiments of a multi-step method, at least one of the first transformation and the second transformation may convert the photoresponsive material from an active to an inactive form. In some embodiments, at least one of the first transformation and the second transformation converts the photoresponsive material from an inactive to an active form. In some embodiments, at least one of the first transformation and the second transformation converts the photoresponsive material from a substantially colorless form to a colored form, or, conversely, from a colored form to a substantially colorless form. In some embodiments, at least one of the first transformation and the second transformation converts the photoresponsive material from a first color to a second color or changes its scattering or absorption properties for light of a given waveband. At least one of the first modified form and the second modified form may be visible under natural light or, alternatively or in addition, at least one of the first modified form and the second modified form may be visible under ultraviolet light. In some embodiments, at least one of the first modified form and the second modified form may be fluorescent material, a phosphorescent material, a polarizing material, a diffracting material, or a refracting material. One or both of the first modified form and the second modified form may be a pigment, dye, pharmaceutical compound, or cosmetic material.

In multi-step methods, registration or alignment of light or photo-responsive materials delivered at different steps may be maintained. A multi-step method may include delivering the second patterned distribution of light in registration with the first patterned distribution of light. The method may include delivering the first patterned distribution of light by placing a first mask over the skin region at a first mask location, the mask including one or more light blocking regions and defining one or more light transmissive regions to form a pattern; and exposing the skin region to light of the first wavelength band. The second patterned distribution of light may be delivered by aiming and focusing light of the second wavelength band at a plurality of locations at the second depth in the skin region according to a second pattern. Alternatively, the second patterned distribution of light may be delivered by placing a second mask over the skin region in registration with the first mask location, the mask including one or more light blocking regions and defining one or more light transmissive regions to form a pattern; and exposing the skin region to light of the second wavelength band. Registration of the second mask with the first mask location may be maintained by positioning the second mask with respect to one or more indicia marked on the skin, illustrated in FIGS. 35A-35C. Alternatively, registration of the masks may be maintained placing the first mask over the skin region at a first mask location by placing the first mask in a mounting device positioned relative to the skin region and placing the second mask over the skin region in registration with the first mask location by placing the second mask in the mounting device, wherein the mounting device may be configured to maintain a correct registration of the second mask with respect to the first mask location, as depicted in FIGS. 34A and 34B.

In some multi-step methods, the first patterned distribution of light may be delivered by aiming and focusing light of the first wavelength band at a plurality of locations at the first depth in the skin region according to a first pattern. Such methods may also include delivering the second patterned distribution of light by placing a mask over the skin region in registration with the first patterned distribution of light, the mask including one or more light blocking regions and defining one or more light transmissive regions to form a pattern; and exposing the skin region to light of the second wavelength band. Alternatively, they may include delivering the second patterned distribution of light by aiming and focusing light of the second wavelength band at a plurality of locations at the second depth in the skin region according to a second pattern.

A multi-step method as depicted in FIG. 37 may include delivering photoresponsive material to at least a skin region of a subject by delivering a photochromic material to at least a skin region of a subject, or it may include delivering photoresponsive material to at least a skin region of a subject by delivering a photodynamic therapy agent to at least a skin region of a subject. It may include delivering photoresponsive material to at least a skin region of a subject by delivering a composite material including one or more of a photodynamic therapy agent or a photochromic material to at least a skin region of a subject.

The first modified form may influence the second transformation of the photoresponsive material at the second depth. The first modified form may influence the second transformation by acting in cooperation with light of the second wavelength band to cause the second transformation of the photoresponsive material at the second depth. Alternatively, the first modified form may influence the second transformation by preventing transformation of photoresponsive material by light of the second wavelength band at the second depth. The first modified form may influence the second transformation by promoting transformation of photoresponsive material by light of the second wavelength band at the second depth, or it may influence the second transformation by inhibiting transformation of photoresponsive material by light of the second wavelength band at the second depth. The first modified form may influence the second transformation within the area of overlap between the first patterned distribution of light and the second patterned distribution of light.

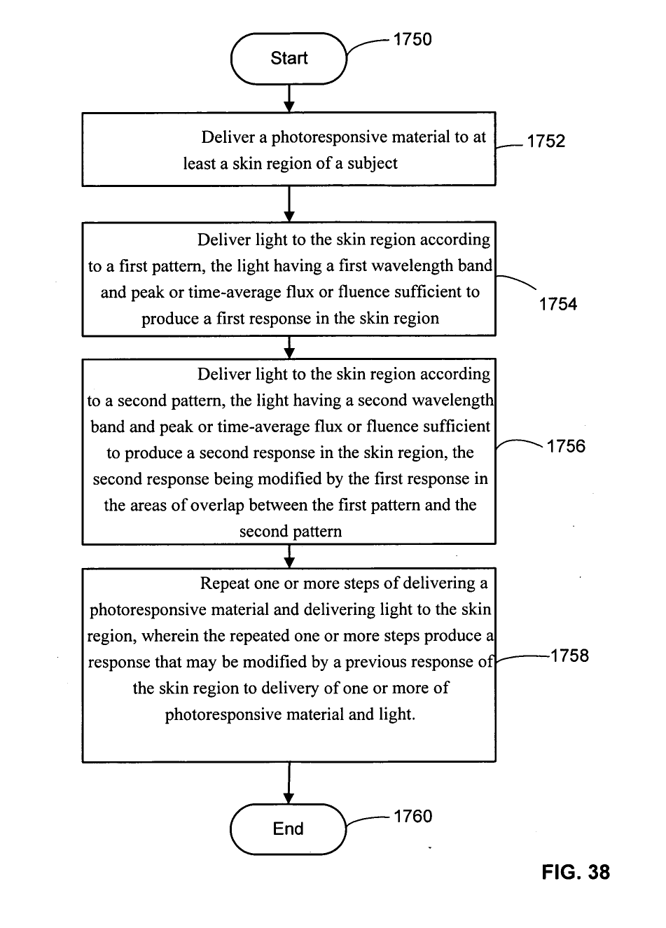

As depicted in FIG. 38, a method of producing a patterned distribution of material in skin, may include the steps of delivering a photoresponsive material to at least a skin region of a subject (step 1752), delivering light to the skin region according to a first pattern, the light having a first wavelength band and peak or time-average flux or fluence sufficient to produce a first response in the skin region (step 1754), delivering light to the skin region according to a second pattern, the light having a second wavelength band and peak or time-average flux or fluence sufficient to produce a second response in the skin region, the second response being modified by the first response in the areas of overlap between the first pattern and the second pattern (step 1756), and repeating one or more steps of delivering a photoresponsive material and delivering light to the skin region, wherein the repeated one or more steps produce a response that may be modified by a previous response of the skin region to delivery of one or more of photoresponsive material and light, as shown at step 1758. Step 1758 of delivering photoresponsive material and delivering light may be repeated in various combinations. The examples of individual method steps and combinations of method steps described and depicted herein are merely exemplary, and based upon disclosure herein a practitioner of skill in the art may devise many different variations.

According to certain embodiments, multi-step photopatterning may be employed to create structures on and above the surface of the skin, within or on top of substrates created or erected on the skin surface. One or more photoresponsive materials may be delivered to the skin surface as described herein. At least the portion of the patterned material formed adjacent to the skin surface may be at least temporarily adherent to the skin surface, or to a substrate material that is adherent to the skin surface. Photopatterning may be performed by delivering targeted or patterned light within a volume of photoresponsive material placed on the surface of the skin. The volume may be defined by the properties (e.g., spreading or adhesion properties) of the photoresponsive material itself, which may be a fluid, gel or paste that will maintain a desired thickness on the skin surface. Alternatively, in embodiments in which the photoresponsive material tends to disperse or spread into too thin a layer, the photoresponsive material may be maintained within a desired area and volume over the skin surface by a retaining enclosure such as a dam or envelope. Such a retaining enclosure may be removed following photopatterning to leave only the patterned structure on the skin surface, or the enclosure may remain in place. For example, the enclosure could have the general appearance of a transparent or translucent patch. Structures on the skin surface having three-dimensional structure may create decorative or cosmetic effects. Three-dimensional structures may include various light-modulating properties that may be modified during steps of forming the three-dimensional structure. Such properties may include light light-absorbing, -reflecting, -scattering, -polarizing, -dispersing, -diffracting, -fluorescing, -phosphorescing or -emitting properties. Three-dimensional structures may have sub-micron feature sizes (i.e., on the scale of wave-lengths of visible light), in order to produce iridescent, opalescent patterning on the skin surface. Alternatively, three-dimensional surface structures may be larger, e.g. to fill or smooth wrinkles, scars, pock marks, and the like, or to modify skin contours, either temporarily, or semi-permanently, to produce an enhanced ‘natural’ appearance or to produce various decorative but not necessarily natural-appearing effects on the skin surface. Three-dimensional surface structures may have structural properties such as rigidity, elasticity, strength, form, shape, bulk, resilience, adhesion or other structural or mechanical properties.

In some embodiments, at least one of the first modified form and the second modified form may be patterned to form a structure with components having a characteristic dimension, spacing, or spatial periodicity of the order of an optical wavelength. Such a structure or pattern may be formed in which at least one of the first modified form and the second modified form includes one or more of a metallic material, a dielectric material, or a resonantly-interacting material. Alternatively, at least one of the first modified form and the second modified form may include a fluorescent, phosphorescent, diffracting, or refracting material. At least one of the first modified form and the second modified form may be patterned to form a structure having a visible appearance that changes as a result of a change of the intensity, color, or incident angle of illuminating radiation or of the angle-of-regard of a viewer.

Systems for delivering patterned light to skin in multi-step methods, for example as described in connection with FIGS. 37 and 38, may be similar to or the same as systems used for delivering patterned light to skin in a single step. Components of such systems may include a first light source capable of producing light of a first wavelength band and peak or time-average flux or fluence, a second light source capable of producing light of a second wavelength band and peak or time-average flux or fluence, a controllable optical system, and electronic circuitry configured to limit the peak or time-average flux and/or fluence of light produced by the light source to levels that are not significantly damaging to the skin at the skin surface. The controllable optical system may be configured to receive a first control signal generated according to a first pattern representing a first desired distribution of light of the first wavelength band and peak or time-average flux or fluence, and to receive a second control signal generated according to a second pattern representing a second desired distribution of light of the second wavelength band and peak or time-average flux or fluence, the controllable optical system responsive to the first control signal to aim and focus light of the first wavelength band at one or more selected skin locations within the first desired distribution, and responsive to the second control signal to aim and focus light of the second wavelength band at one or more selected skin locations within the second desired distribution. Systems may also include various other components, such as memory capable of storing the first pattern and the second pattern in machine readable form, an imaging device, a device driver including one or more of hardware, software, or firmware for generating the control signal based upon pattern data stored in a machine readable medium. In some embodiments of such systems, the first light source and the second light source may be different light sources, in others, the first light source and the second light source may be the same light source. The controllable optical system may include one or more deflectors, which may be configured to aim light from at least one of the first light source and the second light source. The position of at least one of the one or more deflectors may be controllable to aim light toward at least one of the plurality of skin locations.

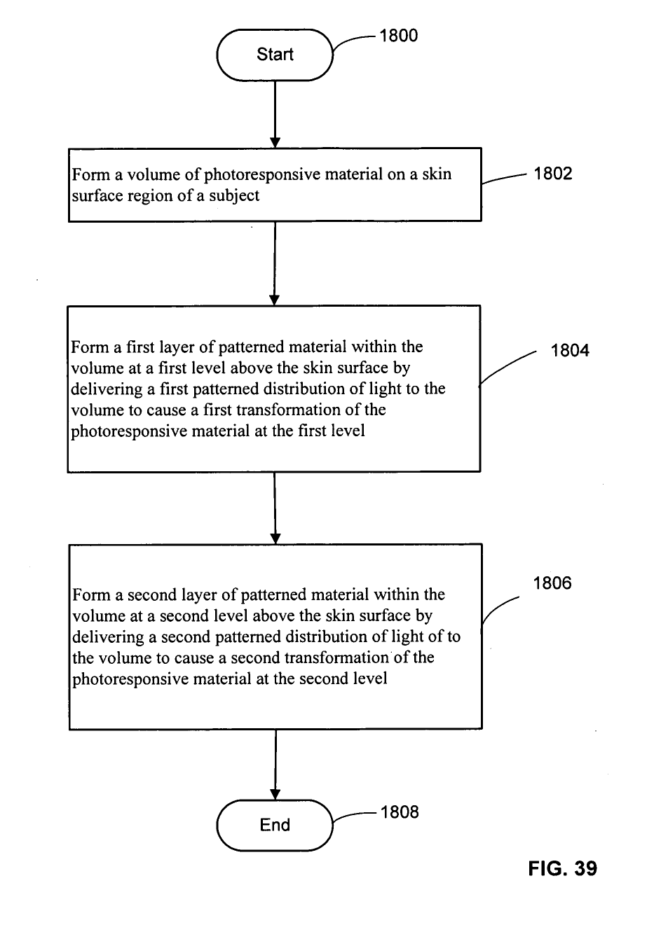

FIG. 39 is a flow diagram of a method of forming a multi-layer structure on skin. At step 1802, a volume of photoresponsive material is formed on a skin surface region of a subject. At step 1804, a first layer of patterned material is formed within the volume at a first level above the skin surface by delivering a first patterned distribution of light to the volume to cause a first transformation of the photoresponsive material at the first level, and at step 1806, a second layer of patterned material is formed within the volume at a second level above the skin surface by delivering a second patterned distribution of light of to the volume to cause a second transformation of the photoresponsive material at the second level. Although a single volume of photoresponsive material is referenced in FIG. 39, more than one volume of photoresponsive material may be used in some embodiments.

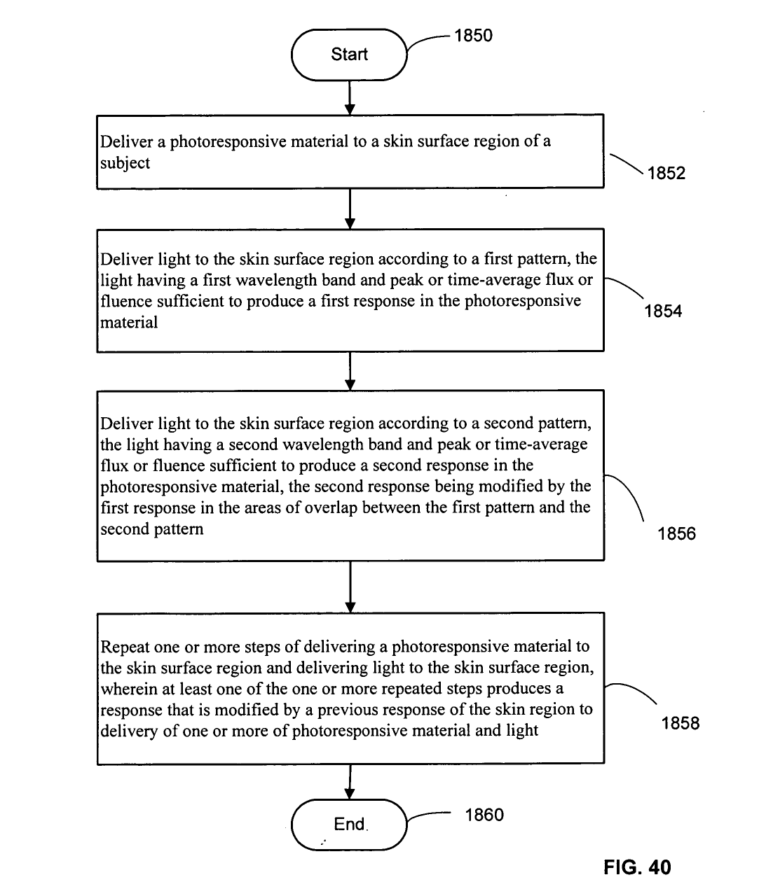

FIG. 40 depicts an exemplary method of forming a patterned distribution of material on a skin surface region. A photoresponsive material is delivered to a skin surface region of a subject at step 1852. At step 1854, light is delivered to the skin surface region according to a first pattern, the light having a first wavelength band and peak or time-average flux or fluence sufficient to produce a first response in the photoresponsive material. At step 1856, light is delivered to the skin surface region according to a second pattern, the light having a second wavelength band and peak or time-average flux or fluence sufficient to produce a second response in the photoresponsive material, the second response being modified by the first response in the areas of overlap between the first pattern and the second pattern. Finally, as indicated at step 1858, one or more steps of delivering a photoresponsive material to the skin surface region and delivering light to the skin surface region may be repeated, wherein at least one of the one or more repeated steps produces a response that is modified by a previous response of the skin region to delivery of one or more of photoresponsive material and light.

FIG. 41 is a flow diagram of a method of modifying a skin surface region. At step 1902, a volume of photoresponsive material is formed on a skin surface region of a subject. At step 1904, a multi-layer structure of a light-modulating material is formed within the volume of photoresponsive material, including forming each layer of the multi-layer structure by delivering a patterned distribution of light to a respective level above the skin surface region within the volume of photoresponsive material, wherein the patterned distribution of light causes transformation of the photoresponsive material to the light-modulating material.

In some embodiments, a volume of photoresponsive material may be formed on a skin surface by applying the photoresponsive material to the skin surface without a dam or other containment structure, for example, in the case where the photoresponsive material is sufficiently thick or viscous that it spreads slowly, if at all, or in the case where a volume of photoresponsive material in excess of the desired volume is applied so that the desired volume may be present on the skin surface even if some of the photoresponsive material flows away. As noted previously herein, in some embodiments a dam may be used to retain a photoresponsive material that would otherwise spread out in a thinner-than-desired layer on the skin surface.

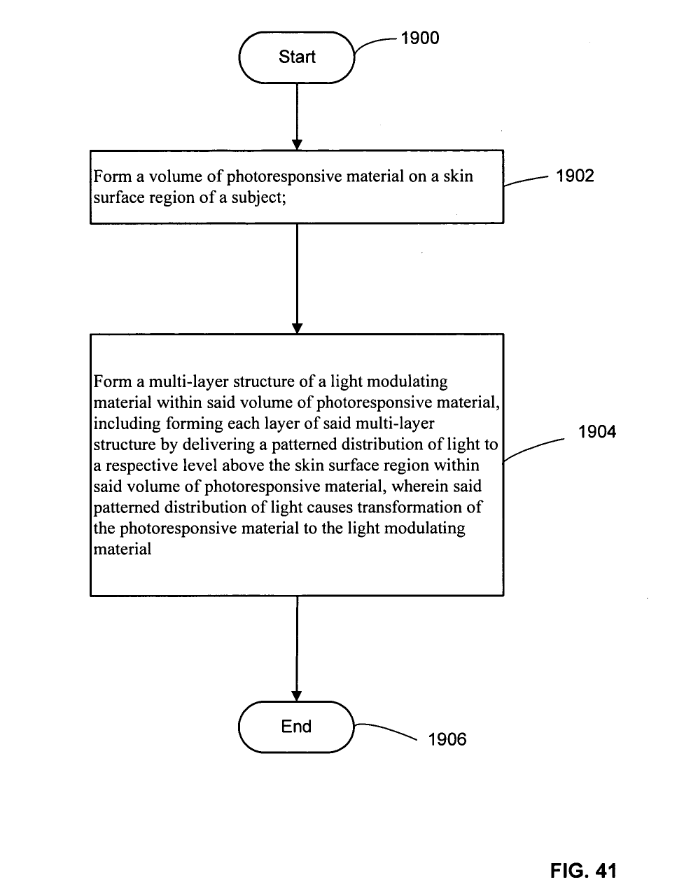

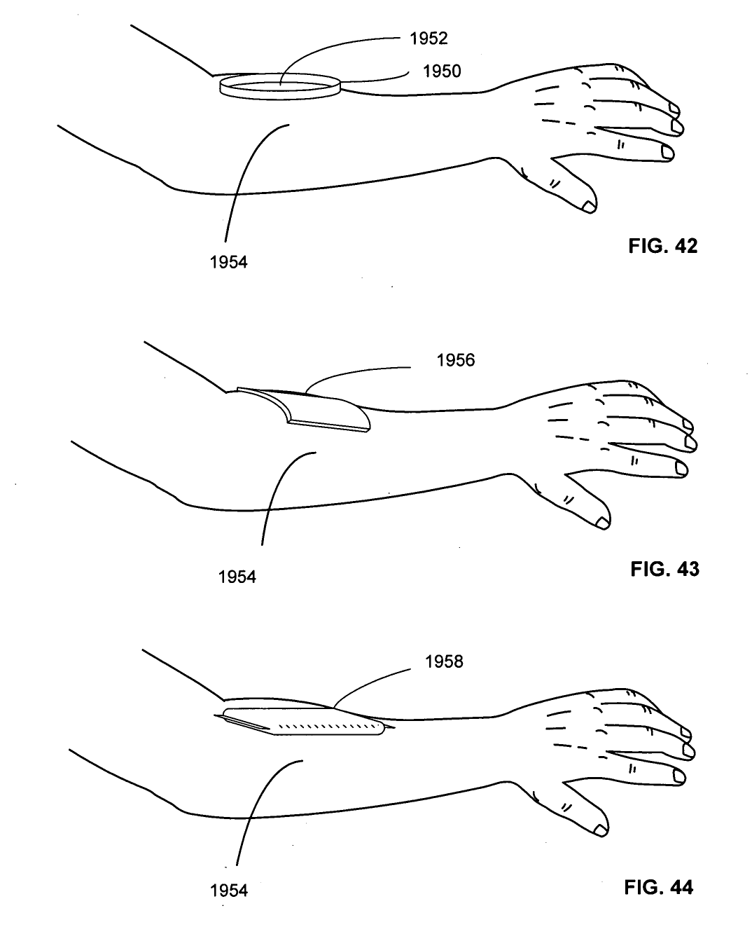

FIG. 42 illustrates a dam 1950 containing a photoresponsive material 1952 on a skin region 1954 of a subject's arm. Dam 1950 may be any type of structure that is capable of being sealed against skin region 1954 sufficiently tightly that photoresponsive material 1952 will be contained within dam 1950. Dam 1950 may be adhered to skin region 1954 with an adhesive or held against skin region 1954 by pressure. In some embodiments, it may be sufficient that dam 1950 simply rest upon skin region 1954. The dam may enclose the skin surface region on which the volume of photoresponsive material is to be formed and have a wall structure sufficient to contain at least one volume of photoresponsive material within the skin surface region.

FIG. 43 illustrates a patch 1956 including a photoresponsive material on a skin region 1954. Patch 1956 may be formed of a matrix material that is impregnated with photoresponsive material, or it may be formed entirely of the photoresponsive material. As in various other embodiments disclosed herein, the photoresponsive material may include a mixture of materials or composite material, of which only certain components respond directly to exposure to light.

FIG. 44 illustrates an envelope 1958 containing a photoresponsive material on a skin region 1954. Envelope 1958 may be, for example, a resilient pouch-like structure that is substantially permeable to light of a wavelength band used to produce modification or transformation of the photoresponsive material. In some embodiments, envelope 1958 may be adhered to the skin surface by an adhesive, or by a self-adhesive property of envelope 1958. In some embodiments, the delivery of light to cause transformation of the photoresponsive material may cause adhesion of at least portions of envelope 1958 to skin region 1954.

FIG. 45A is a cross-sectional view of a dam 2000 containing a photoresponsive material 2002 on a skin surface 2004. Dam 2000 may be generally like dam 1950 as depicted in FIG. 42, i.e., it may be a continuous structure enclosing a region of skin surface 2004. FIG. 45A depicts three-dimensional structures 2006, 2008 and 2010 formed of transformed photoresponsive material 2002. Three-dimension structures 2006, 2008 and 2010 include multiple layers at different levels above skin surface. For example, three-dimensional structure 2006 includes first layer 2012, second layer 2014, and third layer 2016. Similarly, three-dimensional structure 2008 includes first layer 2018, second layer 2020 and three-dimensional structure 2010 includes first layer 2022, second layer 2024, and third layer 2026. After formation of three-dimensional structures by transformation of photoresponsive material 2002, photoresponsive material 2002 and dam 2000 may be removed, leaving three-dimensional structures 2006, 2008, and 2010 on skin surface 2004, as depicted in FIG. 45B. Three-dimensional structures 2006, 2008, and 2010 are merely exemplary of the various three-dimensional structures that may be formed on a skin surface. In different embodiments, different numbers of three-dimensional structures, ranging from a single structure to very large numbers of structures may be formed on a skin surface. Three-dimensional structures may be relatively simple, or complex, both in terms of the numbers of layers from which they are formed as well as the shape and complexity of each layer of each structure.

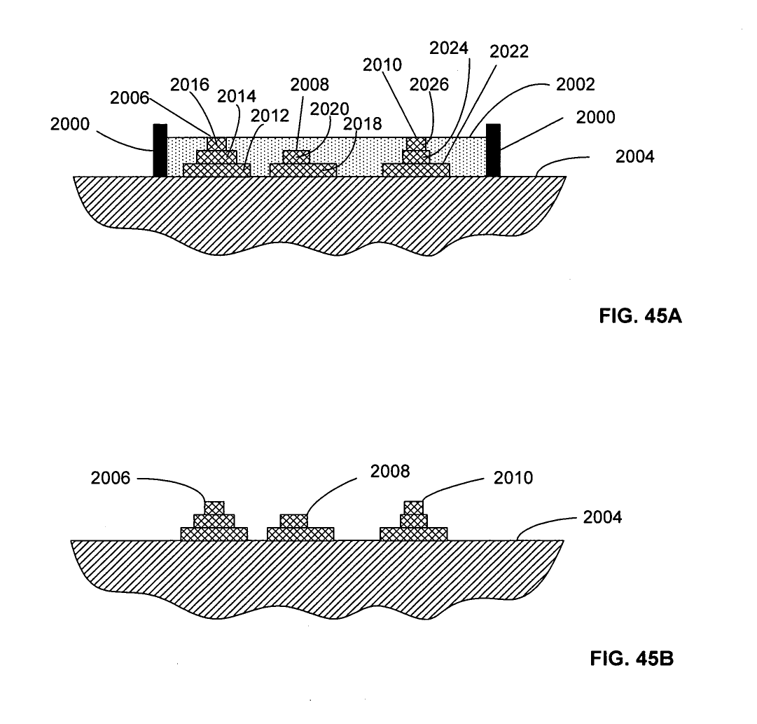

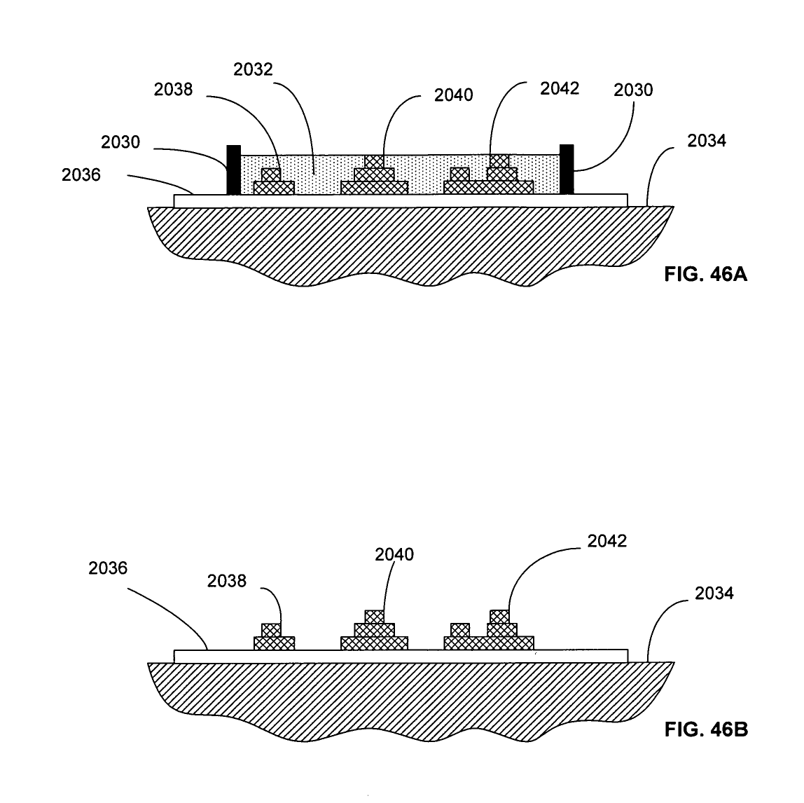

FIG. 46A is a cross-sectional view of another embodiment including a dam 2030 containing a photoresponsive material 2032 on a skin surface 2034. In the embodiment of FIG. 46A, a substrate layer 2036 has been formed on skin surface 2034, and dam 2030 is placed over substrate layer 2036. Three-dimensional structures 2038, 2040, and 2042 may be formed by transformation of photoresponsive material 2032, as described previously, however, in the embodiment of FIG. 46A, three-dimensional structures 2038, 2040, and 2042 are formed on substrate layer 2036. Substrate layer 2036 may provide a smooth surface on which to form three-dimensional structures, improve adhesion between three-dimensional structures and skin surface 2034, or perform other functions such as providing an improved chemical, mechanical, electrical, thermal or light-modulating properties for one or both of the skin surface or the three-dimensional structures formed thereon.

FIG. 46B depicts the embodiment of FIG. 46A following removal of the dam and photoresponsive material. In the embodiment depicted in FIG. 46B, complete substrate layer 2036 remains on skin surface 2034, along with three-dimensional structures 2038, 2040, and 2042. In other related embodiments (not shown in the figures) some portions of a substrate layer may be removed, e.g., the portion of the substrate layer between the three-dimensional structures and the skin surface may be retained and the remainder of the substrate layer removed.

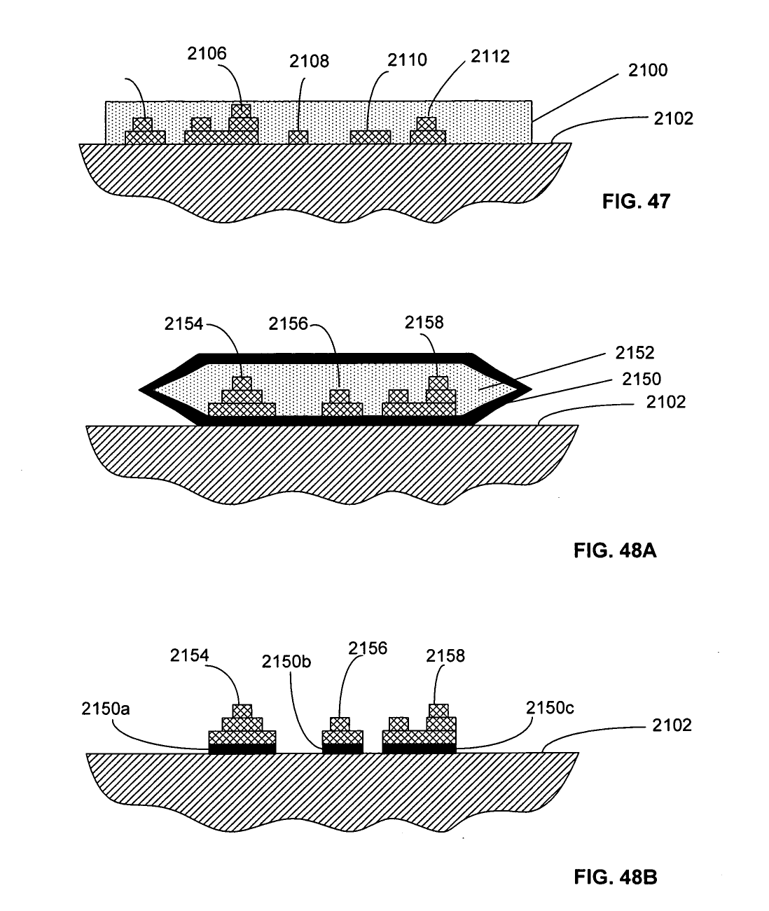

FIG. 47 is a cross-sectional view of a patch 2100 including a photoresponsive material, of the type depicted in FIG. 43, positioned on skin region 2102. Patch 2100 includes three-dimensional structures 2104, 2106, 2108, 2110, and 2112 which have been formed by modification or transformation of photoresponsive material within patch 2100. Patch 2100 may be transparent or translucent, so that three-dimensional structures 2104-2112 may be visible through patch 2100.

FIG. 48A is a cross-sectional view of a skin surface region 2102 on which an envelope 2150 containing a photoresponsive material 2152 has been placed. Three-dimensional structures 2154, 2156, and 2158 are formed within envelope 2150 through modification or transformation of photoresponsive material 2152, as described herein. Following formation of three-dimensional structures 2154, 2156, and 2158, photoresponsive material 2152 and portions of envelope 2150 may be removed, to obtain the structures shown in FIG. 48B. Residual envelope portions 2150a, 2150b, and 2150c, remain between skin surface 2102 and three-dimensional structures 2154, 2156 and 2158, respectively. Portions of envelope 2150 other than residual envelope portions 2150a, 2150b, and 2150c may be removed by treatment with one or more of chemicals, light, etc.

FIG. 49 illustrates an application of formation of three-dimensional structures on a skin surface, for the purpose of smoothing or filling irregularities in or on a skin surface region. FIG. 49 is a cross-sectional view of skin region 2200 having a rough skin surface 2202, which includes both raised areas (e.g., bump 2204) and depressed areas (e.g. depressions 2206 and 2208. A smooth surface 2210 has been formed over skin region 2200 by multi-layer three-dimensional structure 2211, formed by exposure of photoresponsive material to patterned light according to methods described herein. Multi-layer three-dimensional structure 2211 includes layers 2212, 2214, 2216, 2218, 2220 and 2222. As depicted in FIG. 49, uppermost layer 2212 forms a continuous, smooth surface 2210 over skin region 2200, while lower layers 2220 and 2222 are non-continuous and configured to fill recesses, e.g., recesses 2206 and 2208. Intermediate layers 2214, 2216, and 2218 are substantially continuous but include gaps to accommodate raised areas such as bump 2204.

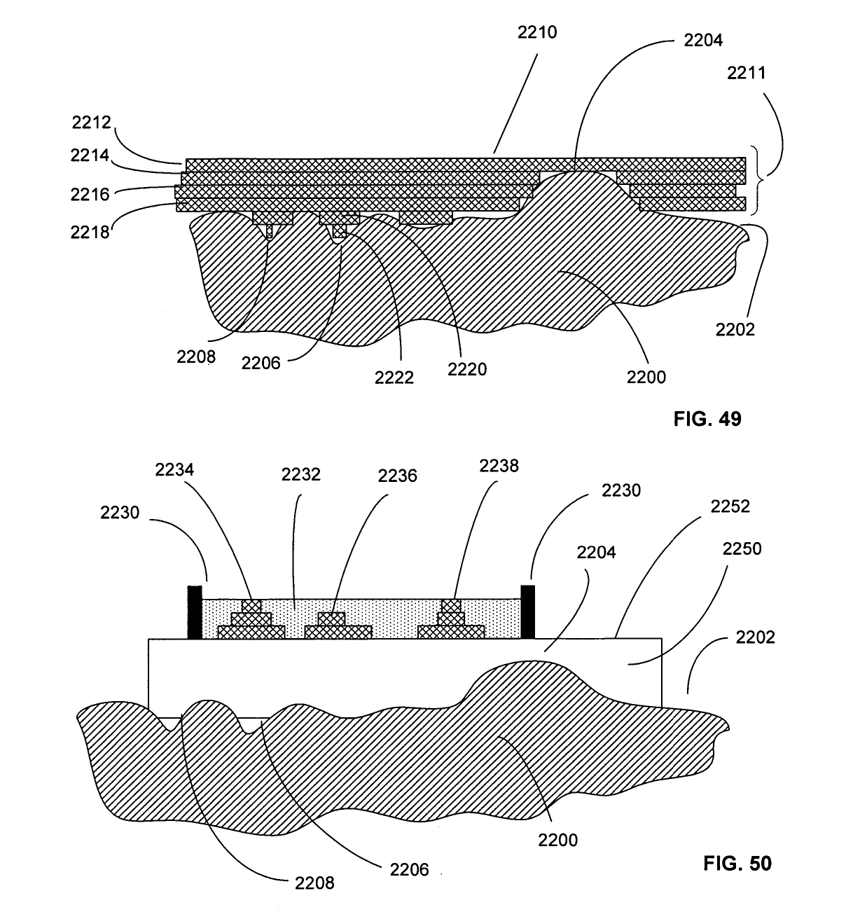

FIG. 50 is a cross-sectional view of a rough skin surface that illustrates the use of a substrate material 2250 to form a smooth surface 2252 over a skin region 2200 having an irregular surface 2202. As shown in FIG. 49, skin surface 2252 includes bump 2204 and depressions 2206 and 2208. Substrate material 2250 may be formed in multiple layers, in the same manner as three-dimensional structure 2211 in FIG. 49, or it may be formed by applying a resilient or flowable material or structure to skin surface 2252 as a single unit. A dam 2230 containing a photoreactive material 2232 may be placed on top of substrate material 2250, and three-dimensional structures 2234, 2236, and 2238 formed from photoresponsive material 2232 as described herein. By providing smooth surface 2252, which may have well-defined light reflecting or absorbing characteristics, beneath three-dimensional structures 2234, 2236, and 2238, the visual or optical effect produced by three-dimensional structures 2234, 2236, and 2238 may be enhanced or otherwise modified. By forming three-dimensional structures on a smoothed surface, the spatial relationship between three-dimensional structures may be controlled.

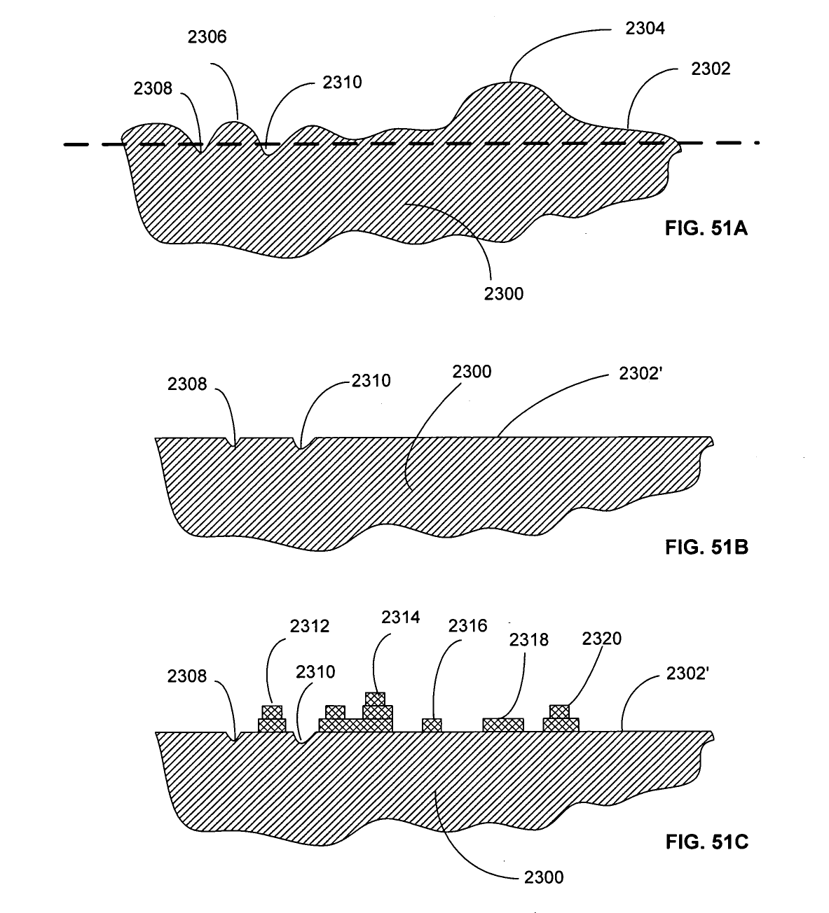

The surface of a skin surface may also be smoothed by various dermo-ablative means prior to application of three-dimensional structures to the skin surface. FIG. 51A is a cross-sectional view of skin region 2300 having a rough surface 2302 that includes raised areas 2304 and 2306 and depressions 2308 and 2310. FIG. 51B is a cross-sectional view of the skin region 2300 following performance of a dermo-ablative process, which might be, for example, a mechanical smoothing or dermabrasion process that removes surface irregularities such as bumps, ridges, etc. The upper layer of the skin (portions above the dashed line in FIG. 51A) have been removed to form smoothed surface 2302′. Smoothed surface 2302′ may be substantially smooth but may not necessarily be completely smooth; for example, portions of depressions 2308 and 2310 may still remain. FIG. 51C is a cross-sectional view of skin region 2300, showing three-dimensional structures 2312, 2314, 2316, 2318 and 2320 erected on smoothed surface 2302′. Three-dimensional structures 2312, 2314, 2316, 2318 and 2320 may be multi-layer structures formed as described previously. By forming three-dimensional structures 2312, 2314, 2316, 2318 and 2320 on smoothed surface 2302′, the visual or optical effect produced by the three-dimensional structures may be enhanced or modified.

With regard to the hardware and/or software used in the control of skin treatment systems according to the present embodiments, and particularly to the sensing, analysis, and control aspects of such systems, those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency or implementation convenience tradeoffs. Those having skill in the art will appreciate that there are various vehicles by which processes and/or systems described herein can be effected (e.g., hardware, software, and/or firmware), and that the preferred vehicle will vary with the context in which the processes are deployed. For example, if an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. For example, those skilled in the art will recognize that optical aspects of implementations will require optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be implicitly understood by those with skill in the art that each function and/or operation within such block diagrams, flowcharts, or examples can be implemented, individually and/or collectively, by a wide range of hardware, software, firmware, or virtually any combination thereof. In one embodiment, several portions of the subject matter subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the capabilities of one of skill in the art in light of this disclosure. In addition, those skilled in the art will appreciate that certain mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory; and transmission type media such as digital and analog communication links using TDM or IP based communication links (e.g., links carrying packetized data).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment).

Those skilled in the art will recognize that it is common within the art to describe devices for detection or sensing, signal processing, and device control in the fashion set forth herein, and thereafter use standard engineering practices to integrate such described devices and/or processes into skin treatment systems as exemplified herein. That is, at least a portion of the devices and/or processes described herein can be integrated into a skin treatment system via a reasonable amount of experimentation.

Those having skill in the art will recognize that systems as described herein may include one or more of a memory such as volatile and non-volatile memory, processors such as microprocessors and digital signal processors, computational-supporting or -associated entities such as operating systems, user interfaces, drivers, sensors, actuators, applications programs, one or more interaction devices, such as data ports, control systems including feedback loops and control implementing actuators (e.g., devices for sensing position and/or velocity and/or acceleration or time-rate-of-change thereof; control motors for moving and/or adjusting components). A skin treatment system may be implemented utilizing any suitable available components, combined with standard engineering practices.

The foregoing-described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected”, or “operably coupled”, to each other to achieve the desired functionality.

While particular aspects of the present subject matter described herein have been shown and described, it will be obvious to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should NOT be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” and/or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of one having skill in the art would understand the convention (e.g., “a system having at least one of A, B, and C” would include but not be limited to systems that have A alone, B alone, C alone, A and B together, A and C together, B and C together, and/or A, B, and C together). In those instances where a convention analogous to “at least one of A, B, or C, etc.” is used, in general such a construction is intended in the sense of one having skill in the art would understand the convention (e.g., “a system having at least one of A, B, or C” would include but not be limited to systems that have A alone, B alone, C alone, A and B together, A and C together, B and C together, and/or A, B, and C together).

Although the methods, devices, systems and approaches herein have been described with reference to certain preferred embodiments, other embodiments are possible. As illustrated by the foregoing examples, various choices of light delivery system configuration and method of delivery of photoresponsive material may be within the scope of the invention. As has been discussed, the choice of system configuration may depend on the intended application of the system, the environment in which the system is used, cost, personal preference or other factors. System design, manufacture, and control processes may be modified to take into account choices of photoresponsive material and intended application, and such modifications, as known to those of skill in the arts of display design and construction, may fall within the scope of the invention. Therefore, the full spirit or scope of the invention is defined by the appended claims and is not to be limited to the specific embodiments described herein.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A method of producing a patterned distribution of material on a skin surface region, comprising:
delivering at least one photoresponsive material to a skin surface region of a subject;
delivering light to the skin surface region according to a first pattern, the light having a first wavelength band and peak or time-average flux or fluence sufficient to produce a first response in the at least one photoresponsive material;
delivering light to the skin surface region according to a second pattern, the light having a second wavelength band and peak or time-average flux or fluence sufficient to produce a second response in the at least one photoresponsive material, said second response being modified by said first response in the areas of overlap between said first pattern and said second pattern; and
repeating one or more steps of delivering a photoresponsive material to the skin surface region or delivering light to the skin surface region, wherein at least one of said one or more repeated steps produces a response that is modified by a previous response of the skin region to delivery of one or more of photoresponsive material and light.

39. The method of claim 38, including delivering light to the skin surface region under microprocessor control.

40. The method of claim 38, including delivering light to the skin surface region according to a first pattern by placing a mask over the skin surface region, the mask including one or more light blocking regions and defining one or more light transmissive regions to form said first pattern; and exposing the skin surface region to said light of a first wavelength band.

41. The method of claim 38, including delivering light to the skin surface region according to said first pattern and delivering light to the skin surface region according to said second pattern in registration.

42. The method of claim 38, including delivering light to the skin surface region according to a first pattern by directing and/or focusing light of said first wavelength band at a first plurality of locations.

43. The method of claim 38, including repeating one or more steps of delivering a photoresponsive material to the skin surface region or delivering light to the skin surface region in order to form thereby a three-dimensional structure from said photoresponsive material on said skin surface region.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A method of modifying a skin surface region, comprising:
forming at least one volume of photoresponsive material on a skin surface region of a subject; and
forming a multi-layer structure of a light-modulating material within said at least one volume of photoresponsive material, including forming each layer of said multi-layer structure by delivering a patterned distribution of light to a respective level above said skin surface region within said at least one volume of photoresponsive material, wherein said patterned distribution of light causes transformation of said photoresponsive material to said light-modulating material.

56. The method of claim 55, including connecting each layer of said multi-layer structure with at least one other adjacent layer of said multi-layer structure to form a three-dimensional structure.

57. (canceled)

58. The method of claim 55, including smoothing said skin surface region prior to forming said at least one volume of photoresponsive material on said skin surface region.

59. (canceled)

60. (canceled)

61. (canceled)

62. (canceled)

63. (canceled)

64. (canceled)

65. The method of claim 55, including connecting or bonding at least one layer of said multi-layer structure to said skin surface.

66. (canceled)

67. (canceled)

68. (canceled)

69. The method of claim 55, including forming a structural property of said multi-layer structure in a separate step from forming a light-modulating property of said multi-layer structure.

70. A method of forming a multi layer structure on skin surface, comprising:
forming a first layer of patterned material within at least one volume of photoresponsive material formed on the skin surface at a first level above the skin surface by delivering a first patterned distribution of light to said at least one volume to cause a first transformation of said photoresponsive material at said first level; and
forming a second layer of patterned material within said at least one volume at a second level above the skin surface by delivering a second patterned distribution of light of to said at least one volume to cause a second transformation of said photoresponsive material at said second level.

71. The method of claim 70, wherein said first level is adjacent to said second level, and wherein at least one of said first transformation and said second transformation causes adhesion or bonding between transformed photoresponsive material of said first level and said second level.

72. The method of claim 70, wherein said first transformation causes at least temporary adhesion or bonding between transformed photoresponsive material of said first level and the skin surface.

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. The method of claim 70, wherein the first transformation influences the second transformation.

90. The method of claim 70, wherein the first transformation of the at least one photoresponsive material includes a conversion of the at least one photoresponsive material from a first state to a second state, and wherein the second transformation of the at least one photoresponsive material includes conversion of the at least one photoresponsive material from a second state to a third state.

91. (canceled)

92. (canceled)

93. The method of claim 90, wherein the at least one photoresponsive material includes two or more components, and wherein the first transformation of the at least one photoresponsive material includes a modification of a first component of the at least one photoresponsive material and wherein the second transformation of the at least one photoresponsive material includes a modification of a second component of the at least one photoresponsive material.

94. (canceled)

95. (canceled)

96. The method of claim 70, including removing untransformed photoresponsive material from said skin surface region following transformation of said at least one photoresponsive material at said first level and said second level.

97. The method of claim 43, wherein at least a portion of said three-dimensional structure produces a decorative or cosmetic effect on said skin surface region; includes one or more structural properties; includes one or more light-modulating properties; has components having a characteristic dimension, spacing, or spatial periodicity of the order of an optical wavelength; includes sub-micron size features adapted to interact with visible light to produce iridescent or opalescent patterning on the skin surface region; or smoothes or fills rough features on said skin surface.

98. The method of claim 38, wherein light delivered to the skin surface region according to said first pattern has at least one of the same wavelength band, peak or time-average flux, or peak or time-averaged fluence as light delivered to the skin surface region according to said second pattern.

99. The method of claim 38, wherein light delivered to the skin surface region according to said first pattern differs from light delivered to the skin surface region according to said second pattern by at least one of wavelength band, peak or time-average flux, or peak or time-averaged fluence.

100. The method of claim 56, wherein connecting each layer with at least one other adjacent layer is performed effectively simultaneously with, subsequent to, or prior to said transformation of said photoresponsive material to said light-modulating material.

101. The method of claim 55, wherein said transformation includes at least one of a photopolymerization reaction, a photo-cross-linking reaction, a photolytic reaction, or a photochromic reaction.

102. The method of claim 65, wherein connecting at least one layer of said multi-layer structure to said skin surface is performed effectively simultaneously with, subsequent to, or prior to said transformation of said photoresponsive material to said light-modulating material.

103. The method of claim 70, wherein at least one of said first transformation and said second transformation includes at least one of a photochromic reaction, a photodimerization reaction, a photopolymerization reaction, a photolytic reaction, a cross-linking reaction, a conversion of said photoresponsive material from a comparatively colorless form to a relatively colored form, or a conversion of said photoresponsive material from a comparatively colored form to a relatively colorless form.

104. The method of claim 70, wherein at least one of said first transformation and said second transformation includes a conversion of said photoresponsive material to a form that includes one or more of a metallic material, a dielectric material, a resonantly-interacting material, a fluorescent material, a phosphorescent material, a polarizing material, a diffracting material, a refracting material, a form that is visible under light visible to the normal human eye, a form that is visible under ultraviolet light, or a form that is visible under infrared light.

105. The method of claim 70, wherein at least one of said first transformation and said second transformation changes at least one of the light reflecting properties of said photoresponsive material, the light scattering properties of said photoresponsive material, or the light absorbing properties of said photoresponsive material.

106. The method of claim 70, including removing untransformed photoresponsive material from said skin surface region following transformation of said photoresponsive material at least one of said first level and said second level.

107. The method of claim 70, including forming at the least one volume of photoresponsive material on a skin surface region of a subject.

108. The method of claim 107, including forming said at least one volume of photoresponsive material on a skin surface region by at least one of delivering said photoresponsive material in the form of an aerosol, cream, emulsion, gel, liquid, fluid, gas, vapor, lotion, patch, powder, or combination thereof; positioning an envelope containing said photoresponsive material on the skin surface region; applying a patch including said photoresponsive material to the skin surface region; or forming a dam on the skin surface, the dam enclosing the skin surface region and having a wall structure sufficient to contain said at least one volume of photoresponsive material within the skin surface region, and forming said at least one volume of photoresponsive material on the skin surface by delivering said photoresponsive material into the skin surface region enclosed by said dam.

109. The method of claim 107, including forming a substrate layer that is effectively adherent to the skin surface and forming at least a portion of said at least one volume of photoresponsive material over said substrate layer.
